(19) United States
(12) Patent Application Publication
(10) Pub. No.: US 2009/0126756 A1
(43) Pub. Date: May 21, 2009

(54) Keratin-protective curl minimizer, compostions, method, and kit therefor

(75) Inventors: Ali Naqi Syed, Oak Brook, IL (US); Narjis Ali Askar, Bartlett, IL (US); Pawel Milczarek, Willow Brook, IL (US)

Correspondence Address: Olson & Cepuritis, LTD., 20 NORTH WACKER DRIVE, 36TH FLOOR, CHICAGO, IL, 60606, US

(73) Assignee: Avlon Industries, Inc.

(21) Appl. No.: 11/990,725

(22) PCT Filed: Sep. 12, 2005
(86) PCT No.: PCT/US05/32410

§ 371 Date: Feb. 20, 2008

(51) Int. Cl. A61Q 5/04; A61K 8/33; A61K 8/362; A61K 8/49; A61K 8/81; A61K 8/73

(52) U.S. Cl. 132/209; 424/70.1; 424/70.11; 424/70.13; 132/202

(57) ABSTRACT

Keratin-fiber protectant, curl minimizing composition are disclosed comprising an effective keratin-protective amount of a physiologically tolerable curl minimizing agent selected from the group consisting of a polyfunctional aldehyde, an activated olefin-containing compound, and a polycarboxylic acid. Also disclosed are methods and kits for using the compositions for minimizing the natural curl configuration of human hair in particular.

### FIELD OF THE INVENTION

This invention relates to removal of the natural curl of hair keratin, and in particular to temporarily loosening or removing some or all of the natural curl of hair while maintaining hair strength.

### BACKGROUND OF THE INVENTION

There is a growing trend and desire by persons having naturally curly hair to alternate, at will, between having a curly or non-curly (i.e., straight) hair style configuration. Thus, there is a growing demand for products that can temporarily remove some or all of the natural curl from the hair to provide the consumer with versatility in styling the hair. There is also an ongoing need for products that can substantially permanently remove some or all of the natural curl from the hair with minimal chemical damage to the hair.

The art of temporarily removing the natural curl from naturally curly hair was practiced, in the early days, by applying a pomade to the hair and then straightening the curl by combing the pomade coated hair, under tension, using a heated metal comb, a process commonly referred to as “pressing.” Although pressing left the hair shiny, silky and temporarily straightened, the process was cumbersome and caused hair damage from excessive heating. The pomades were cosmetically unaesthetic greases and difficult to remove from the hair. Additionally, the metal combs were heated, either electrically or on a stove, to relatively high temperatures, which posed the risk of burning the unprotected skin and scalp or of singeing the hair. More recently, some progress was made using a hair ironing process employing oily lotions and electrical variable heating devices, known as flat hair irons, and hair irons, some of which are combination hair straightener and curling irons, that can be controllably heated. However, the configuration of hair that has been temporarily straightened by these procedures reverts to its natural curly or wavy configuration readily on exposure to humidity, or perspiration, and especially upon washing the hair.

An attempt to achieve temporary hair straightening with some resistance to reversion has been practiced by hairstylists in Brazil using a blow-drying system (known as “escova progressiva”). In this process, solutions of formaldehyde (1.5 to 5%) are used which straighten the hair, which lasts through about two to about three shampooings, or about four weeks, before having to repeat the straightening process. Formaldehyde is known to form cross-links among protein end groups to create a stable, complex matrix. For example, formaldehyde can form cross-links between amide, amino, and tyrosine groups, S—CH₂—NH cross-links between N-terminal cystine and amine groups, NH—CH₂—NH cross-links between amine groups, and S—CH₂—S (djenkolic acid) cross-links.

A variation of the foregoing procedure has been used in Brazil in which the hair is first swollen with a composition, referred to in the trade as a dilator, having a pH of about 8.5, rinsed, then treated with a lotion called a “no frisse lotion” containing 0.2% formaldehyde, along with a thermal protector lotion containing silicones, and the hair is then blow dried and flat ironed. The hair treated in this manner required repeating the treatment process weekly to progressively attain some level of discernible straightening over a period of four consecutive weeks. Upon stopping usage of the procedure, however, the configuration of the hair reverts to its original wavy or curly pattern after two to three shampooings.

Cross-links produced by formaldehyde are not stable to acid or alkaline hydrolysis, and in aqueous solution, formaldehyde undesirably forms methylene glycol and can react with oxygen to produce formic acid. Additionally, exposure to formaldehyde solutions raises health concerns to the users and practitioners, because formaldehyde generally is presumed to be a carcinogen, and can cause contact dermatitis. Thus, formaldehyde solution is toxicologically unacceptable and, in many countries, including Brazil, the amount permitted to be present in hair and skin care products topically applied to humans is controlled and amounts exceeding the allowable level are prohibited. While hair straightened by the foregoing Brazilian processes was silky, straight and shiny, the amount of formaldehyde used exceeds the permissible amount of 0.1% free formaldehyde in cosmetics in Brazil, making it cosmetically undesirable.

Conventional chemical relaxers (hair straighteners) that are formulated to remove substantially all natural curl from the hair are well known in the art and typically contain inorganic or organic bases that convert the disulfide bonds of cystine in hair to stable, irreversible crosslinks of primarily lanthionine by the action of hydroxide ion. These lanthionizing chemical relaxers generally are highly alkaline, typically in the range of about pH 12 to about pH 14 and, and typically produce a straight textural configuration that is substantially permanent (i.e., irreversible). Some consumers, however, do not wish to be committed to just one texture and some are increasingly hesitant to risk exposing their hair to possible damage from such high alkalinity.

An alternative to lanthionization relaxers is a chemical keratin reduction-oxidation relaxer process. Conventional commercial reduction-oxidation processes typically reduce the disulfide bonds in the hair with thiol salts, usually ammonium thioglycolate in a viscous medium at an alkalinity typically in the range of about 9 to about 9.5. The reduced hair is mechanically straightened with combing, and then oxidized with hydrogen peroxide or sodium bromate to restore the disulfide bonds in the new straightened configuration. The straightening effect lasts through about three to four shampooings, but generally provides a less permanent straightening effect than that achieved by lanthionization.

One Japanese attempt to avoid conventional lanthionizing chemical relaxer was a multiple-step, heat-assisted, reduction-oxidation process. The hair was chemically reduced with an ammonium thioglycolate cream composition, shampooed, dried with a hair dryer, mechanically straightened with a heated flat iron set at a temperature of about 200° C., then chemically oxidized with a sodium bromate or hydrogen peroxide lotion, dried with a hair drier, and again mechanically straightened with the heated flat iron. This process, however, was tediously long, taking between four and six hours to complete, and chemical damage was not avoided.

There is an ongoing need and desire, therefore, for cosmetically acceptable compositions and convenient practical processes that temporarily remove some or all of the natural curl from naturally curly hair, and provide some resistance to curl reversion on exposure to humidity, perspiration, or washing, and yet provide the user with versatility in alternating between a straight or curly hair style. There is also an ongoing need and desire for an alternative process to highly alkaline lanthionization processes for removing substantially all the curl from naturally curly hair. The present inventions provides such compositions and processes.

### SUMMARY OF THE INVENTION

Aqueous, keratin-protectant, curl minimizing compositions are disclosed that minimize the curl configuration in naturally curly hair keratin by loosening or removing some or all of the natural wave or curl pattern to straighten the hair while substantially retaining desirable hair properties, e.g., strength and sheen. The curl minimization process preferably is heat-assisted to provide a substantially straightened fiber alignment that lasts through at least one washing.

In one aspect, the curl minimizing compositions temporarily remove the natural curl to permit the straightened hair to revert to its original natural curl configuration upon discontinuing use of the curl minimizing composition and process. In another aspect, the curl minimizing compositions remove substantially all the natural curl to provide permanently straightened hair that resists revision to its naturally curly state through multiple washings or until the natural outgrowth of the hair requires another curl minimization procedure.

The keratin-protectant compositions contain an effective curl minimizing amount of a physiologically tolerable, curl minimizing agent capable of forming cross-links with proteinaceous hair keratin. The curl minimizing agent is preferably selected from the group consisting of a polyfunctional aldehyde; an activated olefin-containing compound having at least one double bond bearing at least one electron withdrawing substituent, such as, without limitation, a carboxylic acid, an ester, an amide, an imide, a nitrile, or an anhydride; and a polycarboxylic acid.

Preferred polyfunctional aldehydes are dialdehydes, and, in particular, glutaraldehyde and glyoxal. A preferred activated olefin-containing compound is an ester of maleic acid, and in particular, a polyalkylene glycol ester of maleic acid. A preferred polycarboxylic acid is 1,2,3,4-butanetetracarboxylic acid (BTCA).

In particular, curl minimizing can be achieved within a practical time period. The keratin-protective, curl minimizing compositions retain the desirable properties of the natural intact hair, such as strength, shine, and softness, and substantially maintain the original, natural color or tone of the hair. The keratin-protectant, curl minimizing compositions can be used in heat-assisted procedures either as the primary or sole curl minimizers or in conjunction with reduction-oxidation processes as auxiliary curl minimizers.

The keratin-protectant curl minimizing compositions can beneficially provide the user with the ability to alternate between having a substantially straight hair style and a curly or wavy hair style, as desired, over selected periods of time. The keratin-protectant curl minimizing compositions can provide temporary to permanent curl minimization sufficient to withstand at least one exposure to humidity, perspiration, or washing without reverting to the hair's original natural curl pattern. The heat-assisted, curl minimizing procedure using the keratin-protective, curl-minimizing compositions of this invention provides a convenient and easy-to-use system for versatility in styling naturally curly hair.

### BRIEF DESCRIPTION OF THE DRAWINGS

In the Figures,

FIG. 1 is a scanned image of curl minimized human hair by a procedure of this invention;

FIG. 2 is a Fourier transform of the image of the curl minimized human hair shown in FIG. 1;

FIG. 3 is a graphical representation of the straightening of excessively curly hair to varying degrees over a sequence of four curl minimizing treatments plotted as a function of a calculated straightening factor; and

FIG. 4 is a graphical representation of the measurement of luster (shine) on clean and soiled human hair by digital image analysis as light intensity (luminance) distribution along a selected section of a given tress plotted as a function of position (pixels) along the hair tress.

### DETAILED DESCRIPTION OF PREFERRED EMBODIMENTS

The singular terms “a”, “an”, and “the”, as used in the specification and the appended claims include plural referents unless the context clearly dictates otherwise. The antecedent term “about” applied to a recited range or value denotes an approximation within the deviation in the range or value known or expected in the art from the measurements method.

Keratin-protectant, curl minimizing compositions of this invention contain an effective curl minimizing amount of at least one physiologically tolerable curl minimizing agent that is capable of forming cross-links with at least one protein end group in hard keratin, such as human hair keratin or wool. Preferred embodiments are described for use on human hair keratin herein. The term “curl minimizing agent” as used herein refers to compounds disclosed herein capable of forming a covalent bond or cross-link with a protein end group in human hair keratin, when applied thereto from an aqueous vehicle. The terms “precondition”, “preconditioning”, and grammatical variation thereof, as used herein refer to compositions containing conditioners which are applied to the hair in a first step of a multi-step curl minimizing process, and to hair that has been so conditioned.

The term “curl minimizing” and grammatical variations thereof as applied to compositions and processes of this invention refers to the visible change in fiber alignment and hence decrease in bulk (volume) of naturally curly keratin, by the removal of some (loosening) or all (straightening) of the natural curl or wave configuration in hair keratin. The term “temporary straightening” means that the curl minimization effect sufficiently resists revision to the natural curl configuration pattern on being subjected to humidity, or perspiration or at least one washing. The term “permanent straightening” means that the curl minimization effect resists reversion to the natural curl configuration on being subjected to more than one washing, preferably at least four washings, and more preferably at least eight washings or lasts until the scalp hair grows sufficiently to visibly warrant another curl minimization procedure. The terms “washed” and “washing” include rinsing with water, shampooing or a combination of rinsing with water and shampooing.

The terms “keratin-protectant” and “hair protectant” are used interchangeably as applied to a curl minimizing composition of this invention and refer to the retention of at least one discernible desirable characteristic, such as strength, sheen, smooth tactile character, and retention of original, natural hair color or tone, and the like, that is conventionally associated with the physical, mechanical integrity of natural hair keratin before receiving a curl minimizing procedure with the composition, and, in particular, before receiving a heat-assisted, curl minimizing procedure of this invention. The term “heat-assisted, curl minimizing”, as used herein, refers to the use of a curl minimizing composition of this invention in conjunction with a heating device, such as an electrically heated flat hair iron or hair dryer at some point in the process. Hand-held hair dryers are conventionally used to “blow dry” hair, preferably using an ambient heat setting temperature in the range of about 100° C. to about 108° C., more preferably in the range of about 105° C. to about 106° C. Commercial electric flat hair irons are available having variable heat settings, and preferably are ceramic, heated to platen temperatures in the range of about 100° C. to about 200° C., more preferably in the range of about 150° C. to about 160° C.

Keratin-protectant, curl minimizing compositions and processes of this invention avoid the deleterious action that can be caused by conventional highly alkaline lanthionization chemical hair relaxer processes, as determined by measured changes in the physical, mechanical integrity of the keratin fibers. Measurable changes in the integrity of the hair include changes in tensile strength properties, elasticity, porosity, cuticle erosion, fiber breakage, wet and dry combing force and the like, as well as changes in discernible subjective properties, i.e., tactile feel, and visible sheen or appearance.

Hard keratin fibers, such as human hair and wool, have a complex morphological structure and are predominately proteinaceous. The proteins in human hair, for example, are polymers made up of a number of amino acids and the linkages of the amino acids and peptide bonds are responsible for the strength of the protein backbone. Hair fibers are quite strong and strength is conferred on the fiber by the inner cortex and protective outer cuticle sheath. The main bulk of the fiber, the cortex, consists essentially of long coiled peptide chains joined in one plane by salt and sulfur linkages, and in the other plane by hydrogen bonds. The chemical structure of hair keratin makes it susceptible to attack by chemical reagents. Hair keratin is more susceptible to alkaline hydrolysis than to acid hydrolysis. A detailed discussion of the reactivity of hair keratin is found in Whewell, “The Chemistry of Hair,” Journal of the Society of Cosmetic Chemists, 15, 423-436 (1964), incorporated herein by reference.

Naturally occurring covalent cross-links in hair keratin contribute to the physical stability and mechanical strength of the fibers. Some naturally occurring, covalent cross-links in the hair include disulfide (cystine) bonds formed between two cystine residues in either the same protein chain or adjacent protein chains; isopeptide bonds formed as amide cross-links in the follicle; and peptide bonds existing between the amino groups of lysine and the carboxyl groups of aspartic acid or glutamic acid in portions of the same polypeptide chain or between different polypeptide chains. The tensile properties of wet keratin fibers are related to the presence of disulfide bonds and the tensile properties of dry keratin are influenced by the peptide bonds. The presence of cross-links in human hair introduced by chemical reagents has been measured in the art directly by analytical techniques, and indirectly, based on changes in the mechanical strength and/or physical stability of the fiber measured instrumentally or quantitatively from such fiber properties as swelling, sorption, super contraction, setting, tensile strength, weight gain, alkali solubility and the like.

The reaction between hair and chemical reagents is generally complex and more than one type of linkage is affected. Naturally occurring sites and linkages that are susceptible to reaction with simple chemical reagents, for example, include hydrogen bonds and van der Waals forces between adjacent peptide chains and side chains of the alpha-keratin-fibers, and salt linkages that can be reversibly fissioned by acids or short time exposure to base. Temporary hair styles that rely on bonding through salt linkages and hydrogen bonds typically are broken by exposure to water and humidity resulting in loss of temporary hair set, typically seen as reversion of a hair style set to its natural configuration, i.e., revert to a curly state (rekink or frizz) or uncurl to a naturally straight state.

The keratin-protectant, curl minimizing compositions of this invention employ curl minimizing agents that can form cross-links with protein end groups in naturally curly keratin to achieve hair styles having a decreased bulk in hair volume by loosening or straightening fiber alignment.

Keratin-protectant, curl minimizing agents of this invention are preferably selected from physiologically tolerable compounds, such as a polyfunctional aldehyde, an activated olefin-containing compound or a polycarboxylic acid, known in the art to bond or form cross-links with at least a protein end group of proteinaceous keratin. The term “polyfunctional aldehyde” means more than one aldehyde is present in the compound, such as a dialdehyde, a trialdehyde, and the like.

Preferred polyfunctional aldehydes are dialdehydes, such as glutaraldehyde and glyoxal, present in physiologically tolerable amounts in the range of about 0.1 to about 3 weight percent, preferably in the range of about 0.15 to about 2 weight percent, more preferably in the range of about 0.2 to not more than about 0.5 weight percent, on a total composition weight basis. Reportedly, one or both functional groups of dialdehydes can react with protein end groups to produce a more complex, more cross-linked protein-aldehyde product with a higher molecular weight that is more stable (i.e., less readily hydrolyzed) than complexes formed by the monofunctional aldehyde formaldehyde. References to “formaldehyde” herein are used for convenience to mean “free” formaldehyde obtained from formalin.

Preferably, glutaraldehyde is used in combination with hydrolyzed polyvinyl alcohol (PVA). PVA reportedly reacts with glutaraldehyde by acetalization to form a bulky pentane dilated PVA having highly reactive aldehyde, hemiacetal, and acetal groups available for reaction with the amino acids of hair containing OH groups, such as serine, tyrosine and threonine, to form a crosslinking network. The reported percentage of serine in hair is relatively high (11.52%) and is comparable to half cystine hence serine can be easily targeted for crosslinking. Any free PVA present in the hair, which has not reacted with the dialdehyde can remain in the matrix of the fiber, and further help in straightening due to its high molecular weight. Any free glutaraldehyde left after the acetalization reaction can also crosslink the hair.

Also preferred, are combinations of glutaraldehyde or glyoxal with Lewis acid catalysts, such as zinc nitrate (Zn(NO₃)₂.6H₂O) aluminum sulfate (Al₂(SO₄)₃), magnesium chloride (MgCl₂.6H₂O), aluminum chloride (AlCl₃), and the like. Polyfunctional aldehydes can react with hydroxyl groups to form a hemiacetal, which can be catalyzed by a Lewis acid to form a stable acetal. Crosslinking by hemiacetal is hydrolyzed easily by water whereas acetal crosslinks are stable under neutral and alkaline conditions. Preferred catalysts are zinc nitrate and magnesium chloride.

An activated olefin-containing compound has at least one double bond bearing at least one electron withdrawing substituent, such as, without limitation, a carboxylic acid, an ester, an amide, an imide, a nitrile, or an anhydride. Preferably, the physiologically tolerable, activated olefin-containing compound, is substantially non-irritating to human skin, is substantially chemically stable in the composition medium, and does not itself impart, or contribute to, any visibly undesirable coloration on the hair or skin. Preferred activated olefin-containing compounds include maleic acid and esters thereof, maleimides and N-substituted derivatives thereof, maleamic acid and N-substituted derivatives thereof, and maleic anhydride. Preferred N-substituents are N—(C₁-C₂₀) alkyl, N-aryl, and N-haloaryl. Maleic acid derivatives, such as maleimides, are known to undergo nucleophilic addition reactions with thiol groups in wool, and also cross-link amine, hydroxyl, and amide groups.

Preferred are mono-esters of maleic acid, and a preferred mono-ester is a poly(C₂-C₄)alkylene glycol ester. Preferred polyalkylene glycol esters include polyethylene glycol esters, polypropylene glycol esters, polybutylene glycol esters, and combinations thereof. Particularly preferred is a polyalkylene glycol bis-maleinate, sold commercially as a water miscible, medium-viscosity, liquid under the trade name MIRALAN™ HTP, by Ciba Specialty Chemicals Corporation. According to the supplier's data sheets, MIRALAN™ HTP contains about 65 to about 75% by weight poly(oxy-1,2-ethanediyl)-alpha-hydro-omega-hydroxy, (2Z)-2-butenedioate corresponds to CAS Number 37310-95-5, has a pH in the range of about pH 2 (5% solution) to about pH 2.7 (1 gram/liter), and is reportedly anionic in character.

Without limitation, exemplary N-substituted maleimide derivatives include N-naphthylmaleimide, and N-trifluorophenylmaleimide; and an exemplary N-substituted maleamic acid derivative is N-trifluorophenylmaleamic acid.

The amount of hair-protectant, activated olefin-containing compound present in a hair-protectant composition may vary, without limitation, in a range of about 1 to about 50 weight percent, preferably in a range of about 3 to about 40 weight percent, more preferably in a range of about 5 to about 15 weight percent on a total composition weight basis.

Useful polycarboxylic acids, include 1,2,3,4-butanetetracarboxylic acid (BTCA), 1,2,3,4-cyclopentanetetracarboxylic (CPTA), citric acid, polyacrylic acid, and the like. Preferably, polycarboxylic acid is used in combination with a weak base catalyst, such as monobasic sodium dihydrogen phosphate (i.e., NaH₂PO₄), dibasic sodium hydrogen phosphate (Na₂HPO₄) or sodium hydrogen phosphate (NaHPO₄). Also preferred is a combination of polycarboxylic acid and cyclodextrin, particularly a combination of BTCA and cyclodextrin, which reportedly forms a copolymer that can cross-link through amide groups, based on studies with wool. A useful amount of polycarboxylic acid is in the range of about 1 to about 25 weight percent, preferably in the range of about 5 to about 20 weight percent on a total composition weight basis.

Hair-protectant, curl minimizing compositions preferably have a pH value in the range of about pH 2 about pH 11, more preferably in the range of about pH 2.5 to about pH 10.5, most preferably in the range of about pH 4 to about pH 9.

Hair-protectant, curl minimizing compositions of the present invention preferably contain the curl minimizing agent in a cosmetically acceptable aqueous vehicle suitable for application at least once to human hair. Preferably, the hair-protectant compositions also contain conventional cosmetic hair conditioning ingredients, botanical products, and other optional cosmetic ingredients, additives, products or materials, and cosmetic adjuvants, well known in the hair care and personal care formulation arts. For heat-assisted, curl minimizing processes, the aqueous curl minimizing composition preferably contains an auxiliary hair protectant, heat-protective ingredient, such as a silicone or silicone derivative.

Cosmetic ingredients that can be employed in the hair-protectant compositions and system discussed herein are referred to by their commonly used chemical or trade names or by the international nomenclature, commonly referred to as INCI name, designated in any edition of the International Cosmetic Ingredient Dictionary and Handbook, (hereafter INCI Dictionary), such as found in Volumes 1-3, of the Seventh Edition (1997) or Eighth Edition (2000) or Ninth Edition (2002), all published by the Cosmetic, Toiletry, and Fragrance Association, Washington D.C. Numerous commercial suppliers of materials listed by INCI name, trade name, or both, can be found in any edition of the INCI Dictionary and in numerous commercial trade publications, including but not limited to, the 2001 Cosmetic Bench Reference edition of COSMETICS & TOILETRIES®, 115 (13), or the Cosmetic Bench Reference 2004, both published by Allured Publishing Corporation, Carol Stream, Ill. (2001), and the 2001 McCutcheon 's Directories, Volume 1: Emulsifiers & Detergents and Volume 2: Functional Materials, published by McCutcheon's Division, The Manufacturing Confectioner Publishing Co., Glen Rock, N.J. (2001); the relevant disclosures of the INCI Dictionary and each of the foregoing publications being incorporated herein by reference.

The term “cosmetic adjuvant” includes cosmetically useful product finishing and promotional additives, well known and conventionally used in the cosmetic arts to maintain the physical stability of a composition during storage (shelf life), and the visible aesthetic appearance of a composition during storage and during the use of the composition. Cosmetic adjuvants that maintain the stability of products typically include a metal-ion chelating agent, an antioxidizing agent, a preservative, an emulsifying agent, a perfume solubilizer, and the like, but are not limited thereto. Cosmetic adjuvants, sometimes called promotional ingredients, aid in enhancing the aesthetics and marketing appeal of the product and include, without limitation, a product colorant, a fragrance, and the like.

Those skilled in the formulation arts can readily determine the amount of curl minimizing agent to be used in a formulation by the amount of curl minimization desired. The compositions can be prepared and used in the form of aqueous liquids, or formulated as emulsions, by techniques known in the art, containing cosmetically acceptable conditioners and emulsifiers.

Curl minimizing can be achieved by contacting naturally curly hair with an aqueous hair protectant curl minimizing composition for a period sufficient to form covalent cross-links and bonds with available linkages in the hair. When the curl minimizing agent is the primary or sole curl minimizer, the naturally curly hair is preferably precleaned by washing it with a shampoo, followed by blotting of excess moisture with a towel, and optionally air-drying at ambient room temperature until the hair is substantially dry to the touch before applying the curl minimizing composition. When the curl minimizing agent is used in conjunction with a reduction-oxidation procedure, the curl minimizing composition can be applied between the reduction step and the oxidation step, as well as after the oxidation step as described in more detail below. Preferably curl minimizing is heat-assisted as described in the following preferred general method embodiments.

Methods where the Curl Minimizing Agent is the Primary or Sole Curl Minimizer.

In one method aspect, curl minimizing composition containing curl minimizing agent is applied to clean, substantially dry, naturally curly hair, distributed through the hair for a period sufficient to saturate the hair, and then the hair is styled, such as by blow drying with a hand-held hair dryer.

In another method aspect, curl minimizing composition containing curl minimizing agent is applied to clean, substantially dry, naturally curly hair, distributed through the hair and maintained in contact with the hair for a period sufficient to saturate the hair, preferably at least about ten minutes, after which a thermal protectant composition is applied to the hair, distributed therethrough, dried by blow drying the hair with a hand-held electric hair dryer until the hair feels substantially dry to the touch, and then styled with a heated ceramic flat hair iron having a platen temperature of about 160° C. (measured using a HI-9063 microcomputer K Thermocouple, Hanna Instruments).

In a particularly preferred method embodiment, a preconditioning composition, preferably containing curl minimizing agent, is applied to the hair, such as by spraying or brushing, distributed through the hair, and maintained in contact with the hair for a period sufficient to saturate the hair (preferably for at least about five to about 10 minutes). The preconditioned hair is rinsed with tepid tap water for at least about 30 seconds, towel blotted and then contacted with a curl minimizing composition, preferably applied with a brush, distributed through the hair, and maintained in contact with the hair for a period sufficient to saturate the hair (preferably for about at least about five to about 10 minutes). The hair is then contacted with a thermal protectant composition distributed through the hair, the hair is blow-dried using a hand-held electric hair dryer set at high heat (about 105° C.) until the hair feels substantially dry to the touch, and is then styled with a heated flat iron set at a platen temperature of about 160° C. Under practical use (salon) conditions, this entire process can be completed within a practical time period in the range of about 40 to about 75 minutes.

In practicing the methods of this invention, curl minimizing agent also can be present in the preconditioning composition, in the thermal protectant composition, or in both the preconditioning composition and thermal protectant composition, as well as the curl minimizing composition. The preconditioning composition contains a cationic conditioning agent, such as a monomeric or polymeric quaternary ammonium compound and is preferably alkaline when a curl minimizing agent is present. Thermal protectant compositions preferably contain a silicone compound and preferably are slightly acidic to substantially neutral when a curling minimizing agent is present.

In a particularly preferred method embodiment, the preconditioner composition preferably has an alkalinity in the range of about pH 8 to about pH 9.5; the curl minimizing composition preferably has a pH in the range of about pH 2.5 to about 7, more preferably in the range of about pH 4.5 to about 6.5; and the thermal protectant composition preferably has a pH in the range of about pH 4.5 to about 6.5.

### Methods Where the Curl Minimizing Agent is Used in Conjunction With a Reduction-Oxidation Process.

In one preferred method embodiment, the naturally curly hair is first contacted with a viscous keratin-reducing thiol-containing composition, preferably having a pH in the range of about pH 9 to about pH 9.5, for a period sufficient to provide reduced hair, typically in the range of about 30 to not more than about 60 minutes. The keratin-reducing composition is preferably applied to the hair with a brush or comb to distribute the composition through the hair, and the hair is physically smoothed several times during the process. The keratin-reducing composition is then removed by washing the reduced hair with water, and preferably also with shampoo, and the reduced hair is substantially dried by removing moisture with a towel. A curl-minimizing composition, preferably having a pH in the range of about 4.5 to about 6.5, is applied to the substantially dried reduced hair, combed through the hair, and left in contact with the hair for a period of at least about 5 to about 15 minutes to provide reduced, curl-minimized hair. Preferably, during the curl-minimizing process, the hair is covered, such as with a plastic film, and more preferably is also heated with an electric hair dryer. The reduced, curl-minimized hair is then oxidized with a “neutralizing” solution of sodium bromate or hydrogen peroxide, in the form of a lotion, and washed with water. The neutralized, curl-minimized hair is then blow-dried using a hand-held electric hair drier set at high heat, (about 105° C.), until the hair is dry to the touch. The blow-dried hair is then styled with a heated flat hair iron set at a temperature of about 160° C.

In yet another preferred method embodiment where the curl minimizing composition is used in conjunction with a reduction-oxidation process, the same steps are carried out as described above, up to and including the post-oxidation water wash. The washed hair is then contacted with a thermal protectant composition having a pH in the range of about 4.5 to about 6.5, and preferably containing a curl-minimizing agent, and the hair is then blow-dried. The dried hair can then be styled with the heated flat hair iron as previously described.

The technique of styling the hair with heated flat hair irons is well known in the art. Preferably, selected hair tress portions are ironed from the root section to the end section by passing a heated flat hair iron set at a platen temperature of about 160° C. downwardly through the hair.

The effectiveness of the curl minimizing compositions and procedures of this invention in one aspect was measured and numerically expressed by calculating a straightening factor from a modified Fourier transform analysis of the fiber alignment orientation adapted from methods used in the textile art to characterize the orientation of fibrous assemblies. A discussion and description of Fourier transform techniques used to characterize the orientation of fiber alignment and assess its deviation from substantially parallel alignment is given by Pourdeyhimi, B. et al., “Measuring Fiber Orientation in Nonwovens, Part III: Fourier Transform”, Textile Research Journal, 67(2), 143-151 (1997), the disclosures of which are incorporated herein by reference. Fourier transform methods extract fiber orientation information by transforming an image of a fiber assembly from its spatial domain of intensities into a frequency domain with appropriate magnitude and phase values. The frequency form of the image is the new image where gray scale intensities represent the magnitude of the various frequency components, so a higher rate of change in gray scale intensity will be reflected in higher amplitudes.

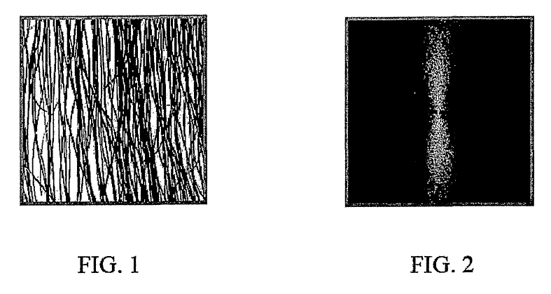

FIG. 1, for example, is a scanned image of excessively curly hair (steam-curled, hair simulating African hair, DeMeo Brothers) that has been straightened by four consecutive curl minimizing treatments of this invention with the combination of compositions for the preconditioner, curl minimizing and thermal protectant and procedure used, and described in more detail in, Example 10 for Tress No. 6. The Fourier transform of the image of FIG. 1 is shown in FIG. 2. A straightening factor can be calculated from the Fourier transform of the image where the straightening factor=1−0.5 width of the maximum of the curves divided by 2πr (which is the circumference of a circle of radius r, where r=50).

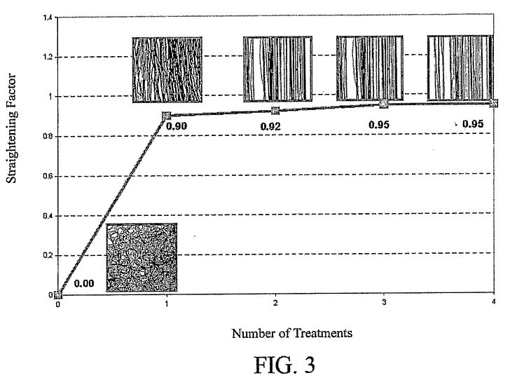

FIG. 3 graphically illustrates the degree of curl (shown in the images) represented from the calculated straightening factor achieved after one (0.90), two (0.92), three (0.95), and four (0.95) consecutive straightening treatments of excessively curly hair (steam-curled hair simulating African hair, DeMeo Brothers) relative to a straightening factor of zero (i.e., untreated hair) with the procedure and combination of compositions for the preconditioner, curl minimizer, and thermal protectant described in Example 10 for Tress No. 6.

It was noted based on subjective visual examination, that hair receiving a curl minimization procedure of this invention was surprisingly lustrous. In another aspect, the efficacy of the curl minimizing efficacy was determined by measuring luster instrumentally and numerically quantifying luster as a method of assessing the keratin protectant effect of the curl minimizing compositions of this invention. The luster of the hair before and after a curl-minimizing procedure was evaluated by analyzing the digital picture of the illuminated tress. The method was a modification of a luster quantification procedure generally described by McMullen et al., “Optical properties of hair: Effect of treatments on luster as quantified by image analysis”, Journal of Cosmetic Science, 54, 335-351 (July/August 2003), the relevant disclosures of which are incorporated herein by reference.

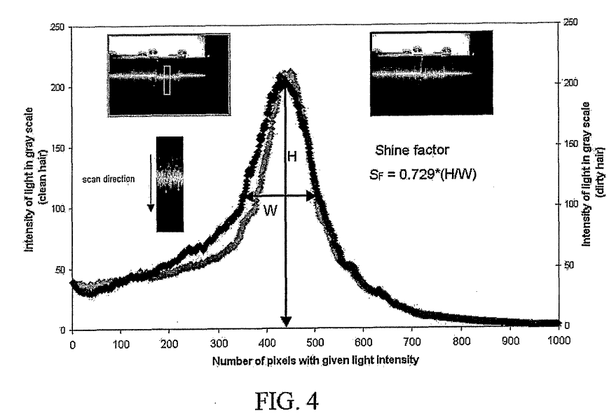

In the modified luster quantification method embodiment, the amount of luster (specular reflection) was measured by photographing the light intensity distribution of hair illuminated with white uncollimated light using a digital camera to produce a gray scale image. Image analysis was carried out using Image Pro 5.1 software. Luster was quantified by calculating a numeric shine factor (Sf) from the height and width of the maximum light intensity (luminance) distribution, as shown in FIG. 4.

FIG. 4 illustrates the measurement of luster on clean and soiled natural black-colored Caucasian hair by digital image analysis. Generally, the hair tress is mounted on a black aluminum cylinder, uniformly illuminated by a beam of white light from a light source, and photographed with a digital camera. The image of clean hair is shown in the upper left corner of FIG. 4 and the image of soiled hair is shown in the upper right corner of FIG. 4.

The analysis of the image is performed by scanning a selected rectangular portion (having 200×800 pixels) judged most representative of the level of shine of the entire tress (region of interest) as illustrated by the image at the upper left of FIG. 4. The selected portion of the image is cropped and sectioned out and the light intensity (luminance) of pixels in gray scale distributed along the vertical axis of the rectangle is determined as shown in FIG. 4. The light intensity (luminance) in gray scale is plotted as a function of number of pixels with given light intensity and a numeric shine factor (Sf) value is calculated by measuring the height (H) and width (W) of the maximum intensity of the specular shine (as shown in FIG. 4 by the arrow on the black curve), determining the value of H/W and multiplying that value by 0.729 (an empirical perfection factor). The black curve represents the specular reflection of “dirty” (i.e., soiled hair) and the gray curve represents the specular reflection of clean hair in FIG. 4.

For evaluating the efficacy of a curl minimizing treatment on shine, the Sf value is determined relative to a Sf value of 1, where Sf 1=maximum luster (shine). An Sf value of at least about 0.6 or higher following a curl minimizing treatment of this invention was judged to correlate with subjectively evaluated visible luster, thus indicating a protectant retention of hair luster. At a Sf value of below about 0.3, the hair was judged to be visibly dull.

The hair protectant efficacy of the curl minimizing compositions of this invention were also evaluated, in yet another aspect, by determining retention of the tensile strength of the hair employing an Intermittent Stress Relaxation (ISR) method, described in the Materials and Method section below.

The keratin-protectant, curl minimizing compositions of this invention are preferably provided in kits in packaged form together with instructional indicia for use. A preferred kit embodiment includes at least one of a hair protectant, a curl-minimizing composition, and a thermal protectant composition. For use in conjunction with reduction-oxidation processes, the kit preferably also contains at least one of a thiol salt-containing, keratin-reducing composition and an oxidative composition. The kit may contain one or more implements for performing a curl minimizing procedure, i.e., disposable gloves, brush, comb, a hand-held blow dryer, a flat hair iron, and the like. The kit components preferably are separately packaged and contained in an outer package. The outer package can be a box or carton or shrink wrap, and preferably has instructional indicia printed thereon or visible therethrough.

Useful instructional indicia can be printed media, aural media, visual aids, electronic media or a combination thereof, which instruct the user on the use of the hair-protectant product. Printed media include, but are not limited to, labels attached to, or imprinted on, the components of the kit, package inserts, pamphlets, books, flyers, and the like. Aural media include, but are not limited to, tape recordings, audio compact disks, records, and the like. Visual aids include, but are not limited, to photographs, slides, movies, videos, DVDs, and the like. Electronic media include all forms of electronic data storage media, such as, but not limited to, diskettes, interactive CD-ROMs, interactive DVDs, and the like.

The following non-limiting examples are provided to further illustrate the invention.

### Materials and Methods

In the following examples the following materials and methods were used, unless otherwise indicated, to assess the efficacy and protectant effect of a hair-protectant, curl minimizing composition of this invention.

### I. Intermittent Stress Relaxation Method (ISR)

The elastic tensile strength of wet hair is measured by determining the Intermittent Stress Relaxation (ISR) of the hair using a Dynamic Mechanical Analyzer (e.g., TA Instruments, Model Q800) equipped with a hair-fiber anchoring assembly that can be submerged in water. An individual hair fiber (about 14.82 millimeters (mm) in length) is clamped at two opposing portions in the fiber anchoring assembly, which is then submerged in water. The length of the submerged hair fiber is then stretched in water to a constant strain of about 0.5% of its length (i.e., an elongation of about 0.07 mm to a total length of about 14.89 mm) for about 30 seconds, and then allowed to relax by removing the strain for about 30 seconds, and this cycle of intermittent stress strain and relaxation is repeated for a total of about 10 cycles.

The amount of stress (in grams/denier fiber) of the hair fiber is measured and recorded before receiving a curl minimizing procedure, and after receiving the curl-minimizing procedure. The ratio (index) of the stress of the hair before receiving the procedure relative to the stress of the hair after the procedure is calculated and the change in the ratio is judged as indicative of loss, or protection from loss, in tensile strength. An index value of 1=no change in hair tensile strength, an index value of less than 1 indicates a loss (weakening) in tensile strength, and an index value of greater than 1 indicates an increase (strengthening) in tensile strength.

Measuring the elasticity of wet hair, when stretched in the range of about 0.5 to about 1 percent of its length, is judged as simulating the range of strain applied during a conventional hair grooming process, such as combing and brushing of the wet hair.

A. Preparation of Fibers for Curl-Minimizing Procedure.

A tress is prepared of Caucasian hair, (naturally brown, De Meo Brothers) about 12 to about 13 cm in length, and about 2 g in weight. About 10 fibers are randomly selected from the same hair lot and the ISR value is determined to provide ISR pre-tested fibers. The ISR pre-tested fibers are then embedded in the tress, anchored at the root portion of the tress. The tress containing the embedded ISR pre-tested fibers is then subjected to the curl minimizing procedure or heat-assisted curl minimizing procedure described below, unless otherwise indicated in the examples. The curl minimized ISR pre-tested fibers are then removed and their individual ISR values again determined as described above. The change in the ratio (index) is determined and a value of at least 1 indicated a protectant effect.

The data were statistically analyzed by computer using SPSS software version 10.1 for WINDOWS. Multiple comparisons of data were statistically performed using one-way Analysis of Variance (ANOVA), post-hoc tukey test, and non-parametric test, such as Sign test at a 95% confidence level.

B. Heat-Assisted Curl Minimizing Procedure for ISR Study

1. Wash tress containing ISR pre-tested fibers once with shampoo (KERACARE® 1ST LATHER® Shampoo, Avon Industries, Inc.).

2. Rinse shampooed tress with tepid tap water for about three minutes, and blot tress with towel to remove excess moisture.

3. Apply preconditioner composition (about 0.5 g), identified in the Example below, preferably by spraying it over tress, distribute through hair with a comb or a brush, and leave in contact with the tress for a period of about 10 minutes to provide a preconditioned tress.

4. Rinse the preconditioned tress with tepid tap water for about one minute, and towel blot to remove excess moisture.

5. Apply a coating of curl minimizing composition (about 0.5 g), identified in the Example below, to the rinsed tress, distribute coating through the hair with a brush, and leave coating in contact with the tress for about 10 minutes.

6. Apply thermal protectant composition (about 0.25 g), identified in the Example below, to the coated tress and substantially blow dry the tress for about three to about four minutes using a hand-held, electric hair dryer set a high heat (about 105° C.) to provide heat-assisted straightened hair.

7. Determine the ISR value of the heat-assisted, straightened, pre-tested fibers.

### II. Assessment of Curl Minimization Efficacy.

Curl minimization efficacy was evaluated by Fourier transform analysis of the fiber alignment distribution and changes in orientation based on a calculated straightening factor as described previously and below, unless otherwise indicated.

A tress of excessively curly hair (kinky) (steam-curled hair simulating African hair, DeMeo Brothers) is prepared weighing about 1.5 g and about 8 centimeters in length. The tress is then treated as follows:

1. Wash tress with shampoo once (KERACARE® 1ST LATHER® Shampoo, Avlon Industries, Inc.), rinse shampooed tress with tepid tap water for about three minutes.

2. Dry the rinsed tress at ambient room temperature (about 25 to about 28° C.) and relative humidity (RH) (about 65% RH).

3. Scan the tress (DELL scanner) and save the image in digital domain.

4. Determine the initial fiber alignment of the digitized image by transferring the image to a computer, opening the image with Image Pro 5.1 software and converting the image to a gray scale.

5. Perform Fourier Transformation (FT) analysis on a selected rectangular section of the gray scale image judged most representative of the overall fiber alignment (i.e., curly or straight).

6. Apply preconditioner composition (about 0.5 g), identified in the Example below, preferably by spraying it on, and leave in contact with the tress for a period of about 10 minutes to provide a preconditioned tress.

7. Rinse the preconditioned tress with tepid tap water for about 30 seconds, towel blot to remove excess moisture.

8. Apply a coating of curl minimizing composition (about 0.5 g), identified in the Example below, to the rinsed tress, preferably with a brush and distribute coating through the hair with the brush, and leave coating in contact with the tress for about 10 minutes.

9. Apply thermal protectant composition (about 0.3 g), identified in the Example below, to the coated tress, manually distribute through the hair, and substantially blow dry the tress for about three to about four minutes using a hand-held, electric hair dryer set at a high heat (about 105° C.) to provide heat-assisted, curl-minimized (straightened) hair.

10. Iron the substantially dried, cooled tress with an electrically heated flat hair iron by passing the heated platen (set at a temperature of about 160° C.) three times through the tress, unless otherwise indicated in the Example below, to provide heat-assisted, curl minimized (straightened) hair.

11. The fiber alignment pattern of the heat-assisted, curl minimized hair is then scanned, digitized, and the image analyzed by FT by repeating the procedures of steps 3, 4, and 5.

12. After a period of at least about 24 hours, the tress is washed once with shampoo and dried by repeating the procedures of steps 1 and 2.

13. The degree of reversion, if any, of the fiber alignment to its original initial alignment pattern is determined by repeating the procedures of steps 3, 4, and 5 at least once to determine the lasting effect of the curl minimizing treatment for a total of two washings, or three times for a total of four washings, or seven times for a total of eight washings (as indicated in the Example below).

The efficacy of curl minimization was determined by calculating a straightening factor from the change in the orientation of the initial fiber alignment pattern after each curl minimizing treatment based on the Fourier transform analysis as described earlier.

The Straightening Factor value generally represented the following fiber alignment: 1=no curl (straight (parallel) fiber alignment); at least about 0.9 to about 0.96=substantially straight (very faint wave) fiber alignment; at least about 0.8 to about 0.89=faint wavy fiber alignment; at least 0.7 to about 0.79=slightly wavy fiber alignment; at least 0.5 to about 0.69=moderately curly to wavy fiber alignment; 0.1 to about 0.49=curly (frizzy); and 0=excessively curly (kinky).

Heat-assisted, curl-minimized hair was judged temporarily straightened if it had a straightened factor in the range of about 0.75 to not more than about 0.8 after four washings, and was judged permanently straightened if it had a straightening factor in the range of at least about 0.8 and about 0.9 after four washings.

### III. Digital Image Analysis of Hair Luster.

A hair tress is prepared of natural brown Caucasian hair (DeMeo Brothers, N.Y.) weighing about 1.5 g and about 8 centimeters in length. The tress is cleaned by washing with commercial shampoo containing anionic surfactant (KERACARE® 1ST LATHER® shampoo, Avlon Industries, Inc.), towel blotted, and air-dried at ambient room temperature in the range of about 25 to about 28° C., and about 65% relative humidity. The cleaned tress is then mounted on an aluminum (painted black) cylinder (10 cm diameter) with clamps at the root and tip sections using the technique described in the discussion of FIG. 2 earlier.

The measurement of shine is conducted on an image of the hair tress taken with a digital camera to record the initial “before treatment” picture and saved in a digital domain. Subsequently, the image is transferred to a computer and opened with Image Pro 5.1 software. The analysis is performed on a gray scale image of the tress using white light by selecting one rectangular section of the image judged most representative of the level of shine of the entire tress. The selected portion of the image is cropped and sectioned out and the intensity of light along the rectangle is measured. The maximum intensity of light is determined and analyzed by measuring the height and half width. An initial shine factor (Sf) value is calculated by measuring the height (H) and width (W) of the curve, determining the value of the H/W and multiplying that value by 0.729 (an empirical perfection factor). The tress is then given a curl-minimizing procedure as described below.

The cleaned tress is sprayed with preconditioning composition (0.6 g), identified in the Example below, left in contact with the hair for about 10 minutes, after which the tress is rinsed with tepid tap water for about 30 seconds, and towel blotted to remove excess moisture. Curl minimizing composition (0.6 g), identified in the Example below, is applied to the blotted tress with a brush and left in contact with the hair for about 10 minutes. After the 10 minutes, thermal protectant composition (0.13 g), identified in the Example below, is applied by hand using the fingers and the tress is blow-dried for about three to about four minutes using a hand-held electric hair drier set at a high setting (about 105° C.). The blow-dried tress is then ironed by passing the tress three times through the heated (about 160° C.) platen of an electrically heated flat hair iron. An after-treatment picture is taken and digitally analyzed as described above, and a final shine factor is calculated as previously described.

The calculated shine factor values are normalized to a value of 1, that is, to “perfectly” shiny hair.

### Example 1

Examples of hair-protectant, curl minimizing compositions containing glutaraldehyde or glyoxal as curl minimizing agents are illustrated in Table 1. General formula 1 (A) provides a preferred range for each curl minimizing agent when present. Specific formulas 1(B-C), contain, on a total composition weight basis, about 0.1% by weight glutaraldehyde, and specific formula 1(D) contains about 0.125% by weight glyoxal, as the curl minimizing agent. The compositions are judged particularly suitable as preconditioning compositions for use in the methods of this invention.

TABLE 1

| | | Parts by Weight (Pbw) As Supplied | | | |
|---|---|---|---|---|---|
| Ingredient | | Ex. 1 (A) | | | |
| INCI/Trade or Common Name | | (Range) | Ex. 1 (B) | Ex. 1 (C) | Ex. 1 (D) |
| 1. | Water, deionized, to 100 pbw | q.s. | q.s. | q.s. | q.s. |
| 2. | Quaternium-80 (40%)(Note 1) | 0.1-0.5 | 0.25 | 0.25 | 0.25 |
| 3. | Polyvinyl alcohol (7.5%) | 0-15 | None | 13.5 | None |
| 4. | Polysorbate 20 | 0.3-1 | 0.6 | 0.6 | 0.6 |
| 5. | Glutaraldehyde (50%) | 0.05-0.3* | 0.2 | 0.2 | None |
| 6. | Hydrolyzed keratin (25%) | 0-1 | 0.5 | 0.5 | None |
| 7. | Zn(NO₃)₂•6H₂O | 0-0.1 | 0.08 | 0.08 | None |
| 8. | Glyoxal (40%) | 0.05-0.4* | None | None | 0.25 |
| 9. | Al₂(SO₄)₃ | 0-0.01 | None | None | 0.007 |
| 10. | MEA** to pH | 0-0.6 | None | None | q.s. |
| 11. | Ammonium hydroxide (28%) to pH | 0-0.5 | q.s. | q.s. | None |
| 12. | Fragrance | q.s. | q.s. | q.s. | q.s. |
| | pH | 8-11 | 8.4 | 10.4 | 10.3 |

Notes to Table 1:
q.s. = quantity sufficient
*Amount when present.
**MEA = Monoethanolamine.
(Note 1) INCI name for quaternary ammonium silicone salt derived from coconut oil sold commercially under the trade name ABIL ® Quat by Goldschmidt, AG.

The compositions may be prepared by formulating techniques well known in the art. A generally preferred procedure for preparing the compositions comprises mixing ingredients nos. 1-4 together at a temperature of about 50° C., cooling and adding ingredients nos. 5-12. The compositions are opaque liquids and remain physically stable after storage at a temperature of about 45° C. for about four months.

### Example 2

Comparative preconditioning compositions containing, on a total composition weight basis, about 0.1% by weight formaldehyde (added as formalin) as a curl minimizing agent are shown in Table 2.

TABLE 2

| | | Parts by Weight (Pbw) | | |
|---|---|---|---|---|
| Ingredient | | As Supplied | | |
| INCI/Trade or Common Name | | Ex. 2 (A) | Ex. 2 (B) | Ex. 2 (C) |
| 1. | Water, deionized, to 100 pbw | q.s. | q.s. | q.s. |
| 2. | Quaternium-80 (40%) | 0.25 | 0.25 | 0.25 |
| | (Note 1, Table 1) | | | |
| 3. | Polyvinyl alcohol (7.5%) | None | 13.5 | None |
| 4. | Polysorbate 20 | 0.6 | 0.6 | 0.6 |
| 5. | Formalin (37%) | 0.27 | 0.27 | 0.27 |
| 6. | Hydrolyzed keratin (25%) | 0.5 | 0.5 | 0.5 |
| 7. | MEA to pH | None | q.s. | q.s. |
| 8. | MgCl₂•6H₂O | None | 0.03 | None |
| 9. | Ammonium hydroxide (28%) | q.s. | None | None |
| | to pH | | | |
| 10. | Fragrance | q.s. | q.s. | q.s. |
| | pH | 8.5 | 10.4 | 10.4 |

The compositions were prepared by admixing ingredient nos. 1-4 together at a temperature of about 50° C., cooling to a temperature of about 30° C., and adding the remaining ingredient nos. 5-10.

All references to formaldehyde and amounts herein and in the remaining Examples refer to “free” or active formaldehyde as calculated from the amount of formalin used.

### Example 3

Preconditioning compositions suitable for use with curl minimizing compositions in the procedures of this invention are shown in Table 3.

TABLE 3

| Ingredient | | Parts by Weight (Pbw) As Supplied | | | | | |
|---|---|---|---|---|---|---|---|
| | INCI/Trade or Common Name | Ex. 3(A) | Ex. 3(B) | Ex. 3(C) | Ex. 3(D) | Ex. 3(E) | Ex. 3(F) |
| 1. | Water, deionized, to 100 pbw | q.s. | q.s. | q.s. | q.s. | q.s. | q.s. |
| 2. | Quaternium-80 (40%) | 0.05 | 0.25 | 0.25 | 0.25 | 0.25 | 0.25 |
| | (Note 1, Table 1) | | | | | | |
| 3. | Polyvinyl alcohol (7.5%) | None | 13.5 | 13.5 | 6.75 | 13.5 | 6.75 |
| 4. | Polysorbate 20 | 0.12 | 0.6 | 0.6 | 0.6 | 0.6 | 0.6 |
| 5. | Hydrolyzed keratin (25%) | 0.1 | 0.5 | 0.5 | 0.5 | 0.001 | 0.5 |
| 6. | TEA* to pH | None | None | None | None | q.s. | None |
| 7. | Ammonium hydroxide (28%) to pH | None | None | q.s. | q.s. | None | q.s. |
| 8. | Cyclodextrin (Note 1) | 1.5 | None | None | None | None | None |
| 9. | Fragrance | q.s. | q.s. | q.s. | q.s. | q.s. | q.s. |

| | | | | | | |
|---|---|---|---|---|---|---|
| pH | 5.2 | 5.1 | 9.5 | 10.5 | 9.5 | 7.5 |

Notes to Table 3:
*TEA = Triethanolamine
(Note 1). INCI name for a beta-cyclodextrin.

The compositions were prepared by admixing ingredient nos. 1-4 at a temperature of about 50° C., cooling to a temperature of about 30° C. and then adding ingredient nos. 5-9.

### Example 4

Curl minimizing compositions containing as curl minimizing agents either glutaraldehyde, glyoxal, 1,2,3,4-butanetetracarboxylic acid (BTCA), or polyalkyleneglycol bis-maleinate (MIRALAN™ HTP) are illustrated in Table 4 (I and II). General formula 4(A) provides a preferred range for each curl minimizing agent (when present). Specific formulas 4(B-E) in Table 4-I and 4(L) in Table 4-II contain glutaraldehyde, specific formula 4(J) contains glyoxal, specific formula 4 (K) BTCA specific formulas 4(F-I) contain a polyalkylene glycol monoester of maleic acid (MIRALAN™ HTP), and specific formula 4(E) contains a combination of MIRALAN™ HTP and glutaraldehyde each in the amounts indicated.

TABLE 4-I

| | | Parts by Weight (Pbw) As Supplied | | | | | |
|---|---|---|---|---|---|---|---|
| Ingredient | | Ex. 4(A) | | | | | |
| INCI/Trade or Common Name | | (Range) | Ex. 4(B) | Ex. 4(C) | Ex. 4(D) | Ex. 4(E) | Ex. 4(F) |
| 1. | Water, deionized, to 100 Pbw | q.s. | q.s. | q.s. | q.s. | q.s. | q.s. |
| 2. | Glycerin, natural (99.5%) | 1-3 | 2 | 2 | 2 | 2 | 2 |
| 3. | Cetrimonium chloride (25%) | 0.1-0.5 | 1 | 1 | 1 | 1 | 1 |
| | (Note 1) | | | | | | |
| 4. | Bisamino PEG/PPG-41/3 | 0.1-0.6 | 0.5 | 0.5 | 0.5 | 0.5 | 0.5 |
| | Aminoethyl PG-Propyl | | | | | | |
| | Dimethicone (30%) (Note 2) | | | | | | |
| 5. | Potassium trideceth-7 | 0.05-0.2 | 0.1 | 0.1 | 0.1 | 0.1 | 0.1 |
| | phosphate (82%) (Note 3) | | | | | | |
| 6. | Quaternium-80 (40%) | 0.1-0.8 | 1 | 1 | 1 | 1 | 1 |
| | (Note 1, Table 1) | | | | | | |
| 7. | Simethicone | 0-0.05 | None | None | None | 0.02 | 0.02 |
| 8. | Laureth-23 | 0.1-0.6 | 0.5 | 0.5 | 0.5 | 0.5 | 0.5 |
| 9. | Propyleneglycol | 0.1-1 | 0.5 | 0.5 | 0.5 | 0.5 | 0.5 |
| | (and) water (and) Camellia | | | | | | |
| | Oleifera Leaf Extract (25%) | | | | | | |
| | (Note 4) | | | | | | |
| 10. | Zn(NO₃)₂•6H₂O | 0-1 | 0.08 | 0.08 | 0.08 | 0.08 | None |
| 11. | Glutaraldehyde (50%) | 0.1-0.5* | 0.4 | 0.2 | 0.2 | 0.4 | None |
| 12. | MgCl₂•6H₂O | 0-0.6 | None | None | None | None | None |
| 13. | Glyoxal (40%) | 0.1-1* | None | None | None | None | None |
| 14. | BTCA | 5-12* | None | None | None | None | None |
| 15. | MIRALAN HTP (Note 4) | 5-50* | None | None | None | 20 | 30 |
| 16. | Sodium lactate | 0-12 | None | None | None | 9 | 9 |
| 17. | Al₂(SO₄)₃ | 0-0.02 | None | None | None | None | None |
| 18. | NaH₂PO₄ | 0-8 | None | None | None | None | None |
| 19. | Fragrance | q.s. | q.s. | q.s. | q.s. | q.s. | q.s. |

| | | | | | | |
|---|---|---|---|---|---|---|
| pH | 2-7 | 5.5 | 5.4 | 5.7 | 4.4 | 4.4 |

TABLE 4-II

| Ingredient | | Parts by Weight (PBW) As Supplied | | | | | |
|---|---|---|---|---|---|---|---|
| INCI/Trade or Common Name | | Ex. 4(G) | Ex. 4(H) | Ex. 4(I) | Ex. 4(J) | Ex. 4(K) | Ex. 4(L) |
| 1. | Water, deionized, to 100 Pbw | q.s. | q.s. | q.s. | q.s. | q.s. | q.s. |
| 2. | Glycerin, natural (99.5%) | 2 | 2 | 2 | 2 | 2 | 2 |
| 3. | Cetrimonium chloride (25%) (Note 1) | 1 | 1 | 1 | 1 | 0.43 | 1 |
| 4. | Bisamino PEG/PPG-41/3 | 0.5 | 0.5 | 0.5 | 0.5 | 0.5 | 0.5 |
| | Aminoethyl PG-Propyl | | | | | | |
| | Dimethicone (30%) (Note 2) | | | | | | |
| 5. | Potassium trideceth-7 | 0.1 | 0.1 | 0.1 | 0.1 | 0.1 | 0.1 |
| | phosphate (82%) (Note 3) | | | | | | |
| 6. | Quaternium-80 (40%) | 1 | 1 | 1 | 1 | 1 | 1 |
| | (Note 1, Table 1) | | | | | | |
| 7. | Simethicone | None | 0.02 | 0.02 | None | 0.02 | 0.02 |
| 8. | Laureth-23 | 0.5 | 0.5 | 0.5 | 0.5 | 0.5 | 0.5 |
| 9. | Propyleneglycol (and) | 0.5 | 0.5 | 0.5 | 0.5 | 0.5 | 0.1 |
| | water (and) Camellia Oleifera | | | | | | |
| | Leaf Extract (25%) (Note 4) | | | | | | |
| 10. | Zn(NO₃)₂•6H₂O | None | None | None | None | None | 0.07 |
| 11. | Glutaraldehyde (50%) | None | None | None | None | None | 0.3 |
| 12. | MgCl₂•6H₂O | None | None | None | None | None | None |
| 13. | Glyoxal (40%) | None | None | None | 0.5 | None | None |
| 14. | BTCA | None | None | None | None | 10 | None |
| 15. | MIRALAN HTP (Note 5) | 10 | 40 | 20 | None | None | None |
| 16. | Sodium lactate | 3.5 | 11 | 7 | None | None | None |
| 17. | Al₂(SO₄)₃ | None | None | None | 0.007 | None | None |
| 18. | NaH₂PO₄ | None | None | None | None | 6 | None |
| 19. | Citric Acid | None | None | None | 0.16 | None | None |
| 20. | Fragrance | q.s. | q.s. | q.s. | q.s. | q.s. | q.s. |

| | | | | | | |
|---|---|---|---|---|---|---|
| pH | 5.1 | 4.1 | 5.0 | 4.1 | 2.8 | 5.7 |

Notes to Tables 4(I-II):
* amount of curl minimizing agent when present.
(Note 1). INCI name for cetyltrimethyl ammonium chloride.
(Note 2). INCI name for a siloxane polymer product, such as sold under the trade name SILSOFT ™ 843 by OSI Specialties.
(Note 3). INCI name for a preneutralized phosphate ester wetting agent sold under the trade name AQUAPHOS ™ TD by Arch Personal Care Industries, L.P.
(Note 4). INCI name for Japanese Green Tea Extract sold under the trade name ACTIPHYTE ® PG 50 by Active Organics.
(Note 5). According to the supplier's data sheets contains about 65%-75% by weight of the polyalkyleneglycol monoester of maleic acid.

The compositions may be prepared by admixing ingredient nos. 1-8 at a temperature of about 50° C., cooling to a temperature of about 30° C., and adding ingredient nos. 9-20. The compositions are opaque liquids and remain physically stable after storage at a temperature of about 45° C. for a period of up to about three to about four months.

### Example 5

Comparative compositions containing, on a total composition weight basis, about 0.2% by weight formaldehyde as a temporary hair straightening agent are shown in Table 5.

TABLE 5

| Ingredient | | Parts by Weight (Pbw) As Supplied | | | | |
|---|---|---|---|---|---|---|
| INCI/Trade or Common Name | | Ex. 5(A) | Ex. 5(B) | Ex. 5(C) | Ex. 5(D) | Ex. 5(E) |
| 1. | Water, deionized, to 100 pbw | q.s. | q.s. | q.s. | q.s. | q.s. |
| 2. | Glycerin, natural (99.5%) | 2 | 2 | 2 | 2 | 2 |
| 3. | Cetrimonium chloride (25%) | 1 | 1 | 1 | 1 | 1 |
| | (Note 1, Table 4) | | | | | |
| 4. | Bisamino PEG/PPG-41/3 | 0.5 | 0.5 | 0.5 | 0.5 | 0.5 |
| | Aminoethyl PG-Propyl | | | | | |
| | Dimethicone (30%) (Note 2, Table 4) | | | | | |
| 5. | Potassium trideceth-7-phosphate | 0.1 | 0.1 | 0.1 | 0.1 | 0.1 |
| | (82%) (Note 3, Table 4) | | | | | |
| 6. | Quaternium-80 (40%) (Note 1, Table 1) | 1 | 1 | 1 | 1 | 1 |
| 7. | Simethicone | None | 0.02 | 0.02 | 0.02 | 0.02 |
| 8. | Laureth-23 | 0.5 | 0.5 | 0.5 | 0.5 | 0.5 |
| 9. | Propylene glycol (and) water (and) | 0.5 | 0.5 | 0.5 | 0.5 | 0.5 |
| | Camellia Oleifera Leaf Extract (25%) | | | | | |
| | (Note 4, Table 4) | | | | | |
| 10. | Formalin (37%) | 0.54 | 0.54 | 0.54 | 12.7 | 3.8 |
| 11. | MgCl₂•6H₂O | 0.5 | 0.5 | 0.06 | 0.06 | 0.06 |
| 12. | Fragrance | q.s. | q.s. | q.s. | q.s. | q.s. |

| | | | | | |
|---|---|---|---|---|---|
| pH | 5.6 | 5.6 | 5.8 | 4.4 | 5.1 |

### Example 6

Thermal protectant compositions containing curl minimizing agents are illustrated in Table 6. Formula 6(A) provides a preferred range for each curl minimizing agent (when present). On a total composition weight basis, specific formulas 6 (B and C) contain about 0.1% by weight glutaraldehyde, and specific formula 6(D) contains about 0.1% by weight glyoxal.

TABLE 6

| | | Parts by Weight (Pbw) As Supplied | | | |
|---|---|---|---|---|---|
| Ingredient | | Ex. 6 (A) | | | |
| INCI/Trade or Common Name | | (Range) | Ex. 6 (B) | Ex. 6 (C) | Ex. 6 (D) |
| 1. | Water, deionized, to 100 pbw | q.s. | q.s. | q.s. | q.s. |
| 2. | Cetrimonium chloride (25%) | 0-2 | 1 | 1 | None |
| | (Note 1, Table 4) | | | | |
| 3. | Quaternium-80 (40%) (Note 1, Table 1) | 0.1-1 | 0.5 | 0.25 | 0.25 |
| 4. | Cetyl alcohol | 3-6 | 4.5 | 4.5 | 4.5 |
| 5. | Ceteth-20 | 1-3 | 1.5 | 1.5 | 1.5 |
| 6. | Simethicone | 0.01-0.1 | 0.05 | 0.02 | 0.05 |
| 7. | Bisamino PEG/PPG-41/3 | 0.1-2 | 1 | 1 | 1 |
| | Aminoethyl PG-Propyl Dimethicone | | | | |
| | (30%) (Note 2, Table 4) | | | | |
| 8. | Dimethicone | 5-20 | 16 | 12.5 | 7.5 |
| 9. | Cyclomethicone | 0-6 | None | None | 5 |
| 10. | Phenyl trimethicone | 0.5-2 | 1 | 1 | 1 |
| 11. | Isostearyl ethylimidazolium | 0.3-0.5 | 0.4 | 0.4 | 0.4 |
| | ethosulfate (Note 1) | | | | |
| 12. | Glutaraldeyde (50%) | 0.05-0.3* | 0.2 | 0.2 | None |
| 13. | Zn(NO₃)₂•6H₂O | 0-0.1 | None | 0.07 | None |
| 14. | Glyoxal (40%) | 0.05-0.4* | None | None | 0.25 |
| 15. | Fragrance | q.s. | q.s. | q.s. | q.s. |
| | pH | 4-8 | 7.1 | 5.9 | 5 |

Notes to Table 6.
*Amount when ingredient is present.
(Note 1) INCI name for a quaternary ammonium salt such as sold under the trade name MONAQUAT ™ ISIES, by Uniqema.

The compositions were prepared by admixing ingredient nos. 1-3 at a temperature in the range of about 70° C. to about 75° C. to provide a water phase. Ingredient nos. 4-11 were admixed in a separate vessel at a temperature in the range of about 70° C. to about 75° C. to provide an oil phase. The oil and water phases are admixed at a temperature of about 75° C. until homogeneous and then cooled to a temperature of about 30° C., and ingredient nos. 12-15 were then added. The compositions are white emulsions, typically viscous lotions having a viscosity (Brookfield) in the range of about 3,000 to about 5,000 mPa·s.

### Comparative Example 7

Comparative thermal protectant compositions containing no curl minimizing agent are illustrated in Table 7 by Formula 7(A). Also included are comparative thermal protectant compositions, Formulas 7 (B-C) containing, on a total composition weight basis, about 0.1% by weight formaldehyde as a temporary hair straightening agent.

TABLE 7

| | | Parts by Weight (Pbw) | | |
|---|---|---|---|---|
| Ingredient | | As Supplied | | |
| INCI/Trade or Common Name | | Ex. 7 (A) | Ex. 7 (B) | Ex. 7 (C) |
| 1. | Water, deionized, to 100 pbw | q.s. | q.s. | q.s. |
| 2. | Cetrimonium chloride (25%) | 1 | 1 | None |
| | (Note 1, Table 4) | | | |
| 3. | Quaternium-80 (40%) | 0.5 | 0.5 | 0.25 |
| | (Note 1, Table 1) | | | |
| 4. | Cetyl alcohol | 4.5 | 4.5 | 4.5 |
| 5. | Ceteth-20 | 1.5 | 1.5 | 1.5 |
| 6. | Simethicone | 0.05 | 0.05 | 0.05 |
| 7. | Bisamino PEG/PPG-41/3 | 1 | 1 | 1 |
| | Aminoethyl PG-Propyl | | | |
| | Dimethicone | | | |
| | (30%)(Note 2, Table 4) | | | |
| 8. | Dimethicone | 16 | 16 | 7.5 |
| 9. | Cyclomethicone | None | None | 5 |
| 10. | Phenyl trimethicone | 1 | 1 | 1 |
| 11. | Isostearyl ethylimidazolium | 0.4 | 0.4 | 0.4 |
| | ethosulfate | | | |
| | (Note 1, Table 6) | | | |
| 12. | Formalin (37%) | None | 0.3 | 0.3 |
| 13. | Fragrance | q.s. | q.s. | q.s. |
| | pH | 7 | 5.7 | 5 |

The compositions are prepared as emulsions generally by the procedure described in Example 6 and are white viscous lotions.

### Example 8

This example illustrates the efficacy of the hair-protectant, curl minimizing compositions and method of this invention, as determined by the Intermittent Stress Relaxation (ISR) Method I described above. The compositions contained polyvinyl alcohol (PVA) in the preconditioner composition. Glutaraldehyde, as the curl minimizing agent, was present at varying concentrations of about 0.1, 0.15 and 0.2% by weight in the curl minimizing composition and at about 0.1% by weight in the thermal protectant composition on a total composition weight basis.

A tress of Caucasian, naturally brown, hair about 2 g in weight and about 12 to about 13 cm in length was prepared as described in Method I(A) above. About 10 fibers were ISR pre-tested as described in ISR Method I(A) and then embedded in the tress. A series of four tresses were prepared. Three tresses (Nos. 1-3) were each separately given a heat-assisted curl minimizing procedure as described in Method I(B) above using the combination of compositions of this invention listed in Table 8 below and the ISR Index was determined. For comparison, the remaining tress (No. 4) was given a similar heat-assisted procedure except that the combination of compositions each containing about 0.1% by weight formaldehyde, listed in Table 8, was used. The ISR Index values are also shown in Table 8.

TABLE 8

| | | CURL- | THERMAL | |
|---|---|---|---|---|
| | PRECONDITIONER | MINIMIZING | PROTECTANT | ISR |
| | FORMULA | FORMULA | FORMULA | INDEX |
| TRESS | | | | |
| NO. | | | | |
| 1 | Ex. 3 (E) | Ex. 4 (L) | Ex. 6 (C) | 1.05 |
| 2 | Ex. 3 (C) | Ex. 4 (B) | Ex, 6 (B) | 1.02 |
| 3 | Ex. 3 (E) | Ex. 4 (D) | Ex. 6 (C) | 1.0 |
| COMPARATIVE | | | | |
| 4 | Ex. 2 (A) | Ex. 5 (B) | Ex. 7 (C) | 1.0 |

Tress No. 1 illustrates glutaraldehyde present at a concentration of about 0.15% by weight in the curl minimizing composition and about 0.1% by weight in the thermal protectant composition, on a total composition weight basis. Tress No. 2 illustrates glutaraldehyde present at a concentration of about 0.2% by weight in the curl-minimizing composition and about 0.1% by weight in the thermal protectant composition, on a total composition weight basis. Tress No. 3 illustrates glutaraldehyde present at a concentration of about 0.1% by weight in each of the curl minimizing and thermal protectant composition, on a total composition weight basis. Comparative Tress No. 4 illustrates formaldehyde present, on a total composition weight basis, at a concentration of about 0.1% by weight in each of the preconditioner compositions and in the thermal protectant composition and about 0.2% by weight in the in the curl minimizing composition. Thus, the cumulative amount of curl minimizing agent in the compositions applied to the hair during the heat-assisted procedure was about 0.25% by weight glutaraldehyde on Tress No. 1, about 0.3% by weight glutaraldehyde on Tress No 2, and about 0.2% by weight glutaraldehyde on Tress No. 3. The total amount of formaldehyde present on Tress No. 4 during the heat-assisted procedure was about 0.3% by weight.

As an added comparison, a fifth Tress (No. 5) was given a similar temporary hair straightener procedure using a commercial product containing about 0.2% formaldehyde. The ISR index was determined to be 0.96 indicating a decrease in hair strength.

Relative to the pre-tested control tress, the ISR index values of Tress Nos. 1 and 2 indicated an increase in hair strength and Tress No. 3 indicated that hair strength was maintained. The ISR index value of comparative Tress No. 4 was comparable to that of Tress No. 3 indicating that, unlike the commercial process, the method of the present invention helped maintain hair strength even when relatively high amounts of formaldehyde were used.

The ISR index value of Tress no. 1 was judged statistically significantly higher (p<0.05) than the ISR index of the commercial product, (using a one-way ANOVA and post-hoc tukey test at a 95% confidence level).

### Example 9

This example illustrates the curl minimizing efficacy of the hair protectant, curl minimizing compositions of this invention as determined by the Digital Image Analysis of Luster Method III described above. A series of four tresses were prepared and an initial shine factor (Sf) value was calculated for each tress. Three tresses (Nos. 1-3) were each given a heat-assisted curl minimizing procedure as described in Method III using the combination of preconditioner, curl minimizing, and thermal protectant compositions of the invention listed in Table 9 below. For comparison, the remaining Tress (No. 4) was given the same heat-assisted procedure of Method III, except that the combination of formaldehyde-containing compositions listed in Table 9 was used.

TABLE 9

| | Preconditioner | Curl Minimizing | Thermal Protectant |
|---|---|---|---|
| | Formula | Formula | Formula |
| Tress | | | |
| No. | | | |
| 1 | Ex. 3 (E) | Ex. 4 (L) | Ex. 6 (C) |
| 2 | Ex. 3 (C) | Ex. 4 (B) | Ex. 6 (B) |
| 3 | Ex. 3 (E) | Ex. 4 (D) | Ex. 6 (C) |
| (Comparative) | | | |
| 4 | Ex. 2 (A) | Ex. 5 (B) | Ex. 7 (C) |

Tress No. 1 illustrates glutaraldehyde present at a concentration of about 0.15% by weight in the curl minimizing composition and about 0.1% by weight in the thermal protectant composition, on a total composition weight basis. Tress No. 2 illustrates glutaradehyde present at a concentration of about 0.2% by weight in the curl minimizing composition and about 0.1% by weight in the thermal protectant composition, on a total composition weight basis. Tress No. 3 illustrates glutaraldehyde present at a concentration of about 0.1% in each of the curl minimizing and thermal protectant compositions, on a total composition weight basis. Comparative Tress No. 4 illustrates formaldehyde present, on a total composition weight basis, at a concentration of about 0.1% by weight in each of the preconditioner and heat protectant compositions and about 0.2% by weight in the curl minimizing composition applied to the hair. Thus, the cumulative amount of curl minimizing agent in the compositions applied to the hair during the heat-assisted procedure was about 0.25% by weight glutaraldehyde on Tress No. 1, about 0.3% by weight glutaraldehyde on Tress No. 2, and about 0.2% by weight glutaraldehyde on Tress No. 3. The total amount of formaldehyde present on Tress No. 4 during the heat-assisted procedure was about 0.3% by weight.

The calculated initial and final shine factor (Sf) value obtained for each tress and % shine retained is shown in Table 9A below.

TABLE 9A

| Tress No. | Sf (initial) | Sf (final) | Retained % Shine* |
|---|---|---|---|
| 1 | 0.75 | 0.69 | 92 |
| 2 | 0.8 | 0.79 | 99 |
| 3 | 0.91 | 0.63 | 69 |
| 4 | 0.95 | 0.5 | 53 |

*Final shine factor relative to initial shine factor.

The Sf data show that Tress Nos. 1-3 treated with curl minimizing compositions of this invention containing glutaraldehyde as the curl minimizing agent were shiny and retained more than 65% of their original luster. In contrast, the luster of the comparative Tress No. 4 decreased considerably. The instrumentally measured shine factor was judged to correlate with subjective luster evaluation.

### Example 10

This example illustrates the curl minimizing efficacy of the hair-protectant, curl minimizing compositions of this invention as determined by Fourier Transform (FT) analysis as described in Method II above.

A series of tresses (Tress Nos. 1-20) were prepared and each one was separately given a heat-assisted, curl minimization procedure as described in Method II using the specific combination of preconditioner, curl minimizing, and thermal protectant compositions listed in Table 10. The efficacy of curl minimization was determined by giving each tress a total of four consecutive curl minimization procedures to simulate a 4-week period, and calculating a straightening factor from the change in initial fiber alignment after each curl-minimizing treatment (CMT) as described in Step 11 of Method II. The degree of curl reversion was also determined by calculating the straightening factor after washing (W) the tress with shampoo, as described in Step 13 of Method II.

Tress Nos. 1-6 illustrate varying amounts of glutaraldehyde as the curl minimizing agent present in both the curl minimizing and thermal protectant composition (Tress Nos. 1, 2, 5, 6) and present in all three compositions (Tress Nos. 3 and 4). Tress No. 7 illustrates glyoxal as the curl minimizing agent present in all three compositions.

Tress Nos. 8 and 9 illustrate BTCA as the curl minimizing agent present in the curl minimizing composition with respectively, beta cyclodextrin and PVA present in the preconditioner.

Tress Nos. 10, 12-15 illustrate different concentrations of polyalkyleneglycol bis-maleinate as the curl minimizing agent present in the curl minimizing compositions.

Tress No. 11 illustrates a combination of glutaraldehyde and polyalkyleneglycol bis-maleinate as the curl minimizing agents in the curl minimizing composition and with glutaraldehyde also present in the preconditioner.

Comparative Tress Nos. 16-20 illustrate the monofunctional formaldehyde as a temporary hair straightening agent in the method of this invention.

TABLE 10

| | | Curl | Thermal |
|---|---|---|---|
| | Preconditioner | Minimizing | Protectant |
| | Formula | Formula | Formula |
| Tress | | | |
| No. | | | |
| 1 | Ex. 3 (D) | Ex. 4 (C) | Ex. 6 (B) |
| 2 | Ex. 3 (D) | Ex. 4 (B) | Ex. 6 (B) |
| 3 | Ex. 1 (C) | Ex. 4 (C) | Ex. 6 (B) |
| 4 | Ex. 1 (B) | Ex. 4 (B) | Ex. 6 (B) |
| 5 | Ex. 3 (C) | Ex. 4 (B) | Ex. 6 (B) |
| 6 | Ex. 3 (E) | Ex. 4 (D) | Ex. 6 (C) |
| 7 | Ex. 1 (D) | Ex. 4 (J) | Ex. 6 (D) |
| 8 | Ex. 3 (A) | Ex. 4 (K) | Ex. 7 (A) |
| 9 | Ex. 3 (B) | Ex. 4 (K) | Ex. 7 (A) |
| 10 | None | Ex. 4 (G) | Ex. 6 (C) |
| 11 | Ex. 1 (C) | Ex. 4 (E) | Ex. 6 (B) |
| 12 | Ex. 4 (H) | Ex. 3 (C) | Ex. 7 (A) |
| 13 | Ex. 4 (F) | Ex. 3 (C) | Ex. 7 (A) |
| 14 | Ex. 3 (B) | Ex. 4 (I) | Ex. 7 (A) |
| 15 | Ex. 3 (A) | Ex. 4 (I) | Ex. 7 (A) |
| Comparative | | | |
| Series | | | |
| 16 | Ex. 2 (C) | Ex. 5 (E) | Ex. 7 (C) |
| 17 | Ex. 2 (C) | Ex. 5 (D) | Ex. 7 (C) |
| 18 | Ex. 3 (D) | Ex. 5 (C) | Ex. 7 (C) |
| 19 | Ex. 2 (B) | Ex. 5 (C) | Ex. 7 (C) |
| 20 | Ex. 2 (A) | Ex. 5 (A) | Ex. 7 (B) |

### Results

The curl minimizing efficacy after each curl minimizing treatment (CMT) and washing (W) obtained with glutaraldehyde as the curl minimizing agent (Tress Nos. 1-6) is shown in Table 10A below.

TABLE 10A
Straightening Factor
After Curl Minimization Treatment (CMT) and Washing (W)

| | First | | | Second | | | Third | | | Fourth | | |
|---|---|---|---|---|---|---|---|---|---|---|---|---|
| | | | % | | | % | | | % | | | % |
| Tress No. | CMT | W | R* | CMT | W | R* | CMT | W | R* | CMT | W | R* |
| 1 | 0.86 | 0.86 | 0 | 0.87 | 0.8 | 8 | 0.87 | 0.84 | 3 | 0.88 | 0.84 | 5 |
| 2 | 0.92 | 0.83 | 10 | 0.95 | 0.84 | 12 | 0.89 | 0.87 | 2 | 0.93 | 0.86 | 8 |
| 3 | 0.91 | 0.82 | 10 | 0.96 | 0.85 | 11 | 0.95 | 0.82 | 14 | 0.95 | 0.83 | 13 |
| 4 | 0.88 | 0.74 | 16 | 0.94 | 0.78 | 17 | 0.9 | 0.81 | 10 | 0.93 | 0.83 | 11 |
| 5 | 0.95 | 0.81 | 15 | 0.95 | 0.86 | 9 | 0.95 | 0.89 | 6 | 0.96 | 0.91 | 5 |
| 6 | 0.9 | 0.86 | 4 | 0.92 | 0.84 | 9 | 0.95 | 0.83 | 13 | 0.95 | 0.84 | 12 |

*% R = Calculated % reversion after washing (W) relative to the CMT.

The data for Tress Nos 1-6 in Table 10A show that, at all times, formulations containing glutaraldehyde as the curl minimizing agent minimized the curl in excessively curly hair to a straightening factor value of at least about 0.8 and retained a straightening factor value of at least about 0.7 after washing. The data also suggest that the presence of polyvinyl alcohol (PVA) in the preconditioner composition (Tress Nos. 1-3, 5 and 6) beneficially promoted curl minimization based on the straightening factor value after washing (compared to Tress No. 4).

The straightening factor obtained with glyoxal as the curl minimizing agent (Tress No. 7) was in the range of about 0.7 to about 0.75 after the first washing and increased to an optimal range of about 0.8 to about 0.85 after the second, third and fourth washing.

The straightening factor obtained with BTCA as the curl minimizing agent (Tress No. 8) was in the range of about 0.1 to about 0.2 after the first, second and third washing and then surprisingly increased to about 0.7 after the fourth washing. Tress No. 9 similarly initially also had a straightening factor in the range of about 0.1 to about 0.2 after the first and second washing then surprisingly increased to about 0.6 and to about 0.8 after the third and fourth washing, respectively. The data from Tress No. 9 also suggested that the presence of PVA in the preconditioning composition promoted curl minimization compared to the data obtained with Tress No. 8.

The straightening factors obtained with Tress Nos. 10-15 with polyalkyleneglycol bis-maleinate as the curl minimizing agent are shown in Table 10 B.

TABLE 10B
Straightening Factor
After Curl Minimization Treatment (CMT) and Washing (W)

| | First | | | Second | | | Third | | | Fourth | | |
|---|---|---|---|---|---|---|---|---|---|---|---|---|
| | | | % | | | % | | | % | | | % |
| Tress No. | CMT | W | R* | CMT | W | R* | CMT | W | R* | CMT | W | R* |
| 10 | 0.9 | 0.71 | 21 | 0.91 | 0.83 | 9 | 0.92 | 0.86 | 11 | 0.92 | 0.87 | 5 |
| 11 | 0.85 | 0.82 | 4 | 0.91 | 0.82 | 10 | 0.92 | 0.79 | 14 | 0.93 | 0.87 | 6 |
| 12 | 0.91 | 0.79 | 17 | 0.89 | 0.79 | 11 | 0.91 | 0.81 | 11 | 0.91 | 0.79 | 13 |
| 13 | 0.88 | 0.75 | 15 | 0.88 | 0.83 | 1 | 0.91 | 0.81 | 11 | 0.88 | 0.79 | 10 |
| 14 | 0.93 | 0.82 | 12 | 0.92 | 0.82 | 11 | 0.91 | 0.88 | 3 | 0.92 | 0.75 | 18 |
| 15 | 0.89 | 0.12 | 87 | 0.88 | 0.15 | 83 | 0.92 | 0.76 | 17 | 0.92 | 0.86 | 17 |

*% R = Calculated % reversion after washing (W) relative to the CMT.

The straightening factor obtained for each of Tress Nos. 10-14 after each treatment was at least about 0.85 after each curl minimizing treatment with compositions containing polyalkyleneglycol bis-maleinate. After each washing the straightening factor was at least about 0.75 for Tress Nos. 10-14. The straightening factor obtained from Tress No. 15 was at least about 0.88 after each curl minimizing treatment. Based on the straightening factor after one to two washings, curl minimizing was judged temporary, decreasing to not more than about 0.15, but after the third to fourth washing, the straightening factor surprisingly increased to at least about 0.75 indicating a cumulative effect when beta-cyclodextrin was present in the preconditioner.

The efficacy of the comparative formaldehyde-containing compositions obtained for Tress Nos. 16-20 is shown in Table 10C below.

TABLE 10C
Straightening Factor
After Curl Minimization Treatment (CMT) and Washing (W)

| | First | | | Second | | | Third | | | Fourth | | |
|---|---|---|---|---|---|---|---|---|---|---|---|---|
| | | | % | | | % | | | % | | | % |
| Tress No. | CMT | W | R* | CMT | W | R* | CMT | W | R* | CMT | W | R* |
| 16 | 0.93 | 0.82 | 12 | 0.93 | 0.86 | 8 | 0.96 | 0.86 | 10 | 0.94 | 0.86 | 9 |
| 17 | 0.93 | 0.85 | 9 | 0.94 | 0.89 | 5 | 0.93 | 0.89 | 4 | 0.93 | 0.87 | 6 |
| 18 | 0.89 | 0.86 | 3 | 0.9 | 0.84 | 7 | 0.88 | 0.84 | 5 | 0.85 | 0.87 | 0 |
| 19 | 0.93 | 0.85 | 9 | 0.95 | 0.85 | 11 | 0.95 | 0.81 | 15 | 0.92 | 0.84 | 9 |
| 20 | 0.89 | 0.55 | 39 | 0.91 | 0.6 | 34 | 0.9 | 0.63 | 30 | 0.93 | 0.72 | 23 |

*% R = Calculated % reversion after washing (W) relative to the CMT.

The comparative data for Tress Nos. 16-19 show that the straightening factor for formaldehyde-containing compositions initially, and after washing, was at least about 0.8. The straightening factor for Tress No. 20 also was at least about 0.8 after each treatment but the straightening factor retained after the first, second and third washings was not more than 0.65, increasing to about 0.72 after the fourth washing. The results obtained with Tress No. 18 suggested that PVA also promoted the efficacy of formaldehyde containing compositions when the method of this invention was used.

### Example 11

This example illustrates the efficacy of using curl minimization compositions of this invention in conjunction with a reduction-oxidation straightening process to augment and enhance the permanency of the hair straightening effect, as determined by digital image analysis of the hair straightening by Method II described above and in curl minimization studies, Study I, and Study II, and comparative Study III described below.

Study I (Heat-Assisted Curl Minimized, Reduction-Oxidation)

A tress of excessively curly hair weighing about 1.5 g was prepared and treated as follows:

Step 1. The tress was washed with shampoo (KERACARE® Hydrating Detangling Shampoo, Avlon Industries, Inc.), blotted with a towel and substantially dried at ambient room temperature and humidity. The fiber alignment was digitally scanned and the Fourier transform image analyzed by the procedure of Method II, as described in Steps 3, 4, and 5.

Step 2. About 6 g of a viscous thiol salt-containing hair-reducing composition (conditioning creme relaxer for normal hair containing about 8.5% ammonium thioglycolate, about 0.4% diammonium dithiodiglycolate, about 1% PVA, as well as conditioners, emulsifiers, and water, and having a pH in the range of about pH 9.2 to about pH 9.3) were applied to the tress and distributed through the hair with a brush, and left in contact with the hair for a period of about 40 minutes, during which time the hair was physically smoothed to a substantially straight configuration twice using the back of a comb, to provide reduced hair.

Step 3. The reduced hair was washed with tepid water for about three minutes to remove the hair-reducing composition, washed with shampoo (KERACARE® Hydrating Detangling Shampoo, Avlon Industries, Inc.), towel blotted, and substantially dried at ambient room temperature and humidity.

Step 4. About 1 g of the curl minimizer composition of Example 4(D) containing glutaraldehyde (about 0.1 weight %) was applied to the reduced hair tress from Step 3 and distributed through the hair with a brush, the tress was wrapped in plastic film, and then heated with an electric hair dryer for a period of about 10 minutes to provide reduced, curl-minimized hair.

Step 5. The plastic film wrap was removed from the reduced, curl-minimized hair, the unwrapped hair tress was laid on a flat plastic sheet, and neutralized by applying about 2 g of a commercial oxidative neutralizer lotion (FERM® Conditioning Neutralizing Lotion, Avlon Industries, Inc., containing sodium bromate and conditioners, and having a pH of about 7), distributing the neutralizer lotion through the hair, and leaving it in contact with the hair for a period of about 15 minutes to provide neutralized, curl-minimized hair.

Step 6. The neutralized, curl-minimized hair tress was thoroughly washed with water, and towel blotted.

Step 7. About 0.8 g of the thermal protectant composition of Example 6(C) containing glutaraldehyde (about 0.1 weight %) was applied to the towel blotted neutralized tress, curl-minimized tress, distributed through the hair, and then the tress was blow-dried with a hand-held electric hair dryer, set at a high heat setting (about 105° C.) until the hair was substantially dry to the touch.

Step 8. The blow-dried hair was then pressed three times with a heated (about 160° C.) ceramic, flat hair iron. The fiber alignment pattern of the tress was again digitally scanned and the Fourier transform image analyzed as described in Method II, Steps 11-13, and the straightening factor was calculated immediately after treatment, and after washing the hair daily as described in Step 1 above for a total of 8 washings with shampoo. The calculated straightening factor values are shown in Table 11, Study I.

Study II (Curl Minimized, Reduction-Oxidation)

A tress of excessively curly hair weighing about 1.5 g was prepared and treated by following the procedure of Study I, except that in Step 4 the wrapped tress was not heated. The calculated straightening factor values are shown in Table 11, Study II.

Study III (Comparative)

A tress of excessively curly hair weighing about 1.5 g was prepared and treated as follows:

Step 1. The procedure of Step 1 of Study I was performed.

Step 2. About 0.3 g of a conditioner (AFFIRM® PROTECTO®, Avlon Industries, Inc., containing a cationic polymer and a pH in the range of about 4 to about 5), was applied to the tress and distributed through the hair.

Step 3. The procedures of Step 2 of Study I was performed to provide reduced hair, except that the viscous, thiol salt-containing hair-reducing composition used was a commercial product of substantially similar strength but containing no PVA (FERM® Conditioning Retexturizing Creme for normal hair, Avlon Industries, Inc., containing about 8.5% ammonium thioglycolate, about 0.4% diammonium dithiodiglycolate, as well as conditioners, emulsifiers, and water, and having a pH in the range of about pH 9.2 to about pH 9.3).

Step 4. The reduced hair was washed with tepid water for about three minutes to remove the hair-reducing composition, washed with shampoo (KERACARE® Hydrating Detangling Shampoo, Avlon Industries, Inc.), and towel blotted.

Step 5. About 0.8 g of conditioner (KERACARE® Leave-In Conditioner, Avlon Industries, Inc.) was applied to the blotted reduced hair from Step 4, and the tress was blow-dried with a hand-held electric hair dryer, set at a high heat setting (about 105° C.) until the hair was substantially dry to the touch.

Step 6. About 0.5 g of a silicone-containing thermal protectant composition (a blend of about one part by weight of KERACARE® Silken Seal (containing silicones) and about one part by weight of FERM® Heat Protection Complex (containing silicone and oils), both available from Avlon Industries, Inc.) was applied to the dry reduced hair, distributed through the hair, and then the tress was pressed by passing the tress three times through with a heated (about 160° C.) flat hair iron.

Step 7. The ironed reduced hair was then neutralized by applying about 2 g of oxidative neutralizer lotion (FERM® Conditioning Neutralizing Lotion, Avlon Industries, Inc., containing sodium bromate and conditioners, and having a pH of about 7) to the hair, distributing the neutralizer through the hair and leaving it in contact with the hair for a period of about 15 minutes.

Step 8. The neutralized tress from Step 7 was thoroughly washed with warm water, and towel blotted.

Step 9. The blotted neutralized tress was then blow-dried with a hand-held electric hair dryer, set at a high heat setting (about 105° C.) until the hair was substantially dry to the touch.

Step 10. The blow-dried hair was then pressed three times with a heated (about 160° C.) ceramic flat hair iron. The fiber alignment pattern of the tress was again digitally and image analyzed determined as described in Method II, Steps 11-13, and the straightening factor was calculated immediately after treatment, and after washing the hair daily as described in Step 1 above for a total of 8 washings with shampoo. The calculated straightening factor values are shown in Table 11, Study III.

TABLE 11
Calculated Straightening Factor of
Tress Relative to Initial = 0

| Stage | Study I | Study II | Study III |
|---|---|---|---|
| Day 1 | 0.96 | 0.94 | 0.92 |
| W - 1 | 0.92 | 0.91 | 0.84 |
| W - 2 | 0.90 | 0.90 | 0.84 |
| W - 3 | 0.90 | 0.90 | 0.84 |
| W - 4 | 0.90 | 0.88 | 0.83 |
| W - 5 | 0.89 | 0.85 | 0.84 |
| W - 6 | 0.91 | 0.84 | 0.83 |
| W - 7 | 0.91 | 0.84 | 0.81 |
| W - 8 | 0.91 | 0.84 | 0.80 |

Note to Table 11:
Day 1 = Straightening factor at the end of the process before the first washing (W). Each washing “W” is identified by the arabic numeral shown.

The data show that surprisingly permanent straightening was achieved and retained through 8 shampoo washings with the curl minimized reduction oxidation processes of Studies I and II, and that heat-assisted curl minimization particularly favored straightening. Relative to the Day 1 data, the reversion after one washing of the hair from the heat-assisted process of Study I was only about 4% and after 8 washings of the hair from the heat-assisted process of Study I was only about 5%. Relative to the Day 1 data, the reversion after one washing of the hair from the process of Study II (where no heat was used during the curl minimization step) was about 3%, increasing to about 6% after 4 washings, and to a maximum range of about 10 to about 11% after 5 washings. In contrast in the comparative Study III (where no curl minimization was used), relative to the Day 1 data, the reversion after one washing was about 7% increasing to about 10% after 4 washings and to a range of about 12 to about 13% after 7 washings.

### Example 12

This example illustrates the efficacy of using curl minimization compositions of this invention in conjunction with a reduction-oxidation straightening process to augment and enhance the permanency of the hair straightening effect, as determined by the Intermittent Stress Relaxation (ISR) Method I as described above and below.

A series of two-gram tresses of Caucasian, naturally brown, hair were prepared as described in Method I(A) above. About 10 fibers were ISR pre-tested as described in ISR Method I(A) and then embedded in each tress. A tress was then treated as described in either Study I, Study II or Study III below:

Study I (Heat-Assisted Curl Minimized, Reduction-Oxidation)

Step 1. About 8 g of a viscous thiol salt-containing, hair-reducing composition (conditioning creme for normal hair containing about 8.5% ammonium thioglycolate, about 0.4% diammonium dithiodiglycolate, about 1% PVA, as well as conditioners, emulsifiers, and water, and having a pH in the range of about pH 9.2 to about pH 9.3) were applied to the tress with a brush, distributed through the hair, and left in contact with the hair for a period of about 40 minutes, during which time the hair was smoothed at least twice with the back of a comb to provide reduced hair.

Step 2. The reduced hair was washed with tepid water for about three minutes to remove the hair-reducing composition, washed with shampoo (KERACARE® Hydrating Detangling Shampoo, Avlon Industries, Inc.), towel blotted, and substantially dried at ambient room temperature and humidity.

Step 3. About 1.2 g of the curl minimizer composition of Example 4(D) containing glutaraldehyde (about 0.1 weight %) was applied to the towel blotted reduced hair tress, distributed through the hair, the tress was wrapped in plastic film, and then heated with an electric hair dryer for a period of about 10 minutes to provide reduced, curl-minimized hair.

Step 4. The plastic film wrap was removed from the reduced, curl-minimized hair, the unwrapped hair tress was laid on a flat plastic sheet, and neutralized by applying about 2 g of a commercial oxidative neutralizer lotion (FERM® Conditioning Neutralizing Lotion, Avlon Industries, Inc., containing sodium bromate and conditioners, and having a pH of about 7), distributing the neutralizer lotion through the hair with a brush, and leaving it in contact with the hair for a period of about 15 minutes to provide neutralized, curl-minimized hair.

Step 5. The neutralized, curl-minimized hair tress was thoroughly washed with water, and towel blotted.

Step 6. About 0.8 g of the thermal protectant composition of Example 6(C) containing glutaraldehyde (about 0.1 weight %) was applied to the towel blotted neutralized tress, distributed through the hair, and then the tress was blow-dried with a hand-held electric hair dryer, set at a high heat setting (about 105° C.) until the hair was substantially dry to the touch.

Step 7. The blow-dried hair was then pressed three times with a heated (about 160° C.) ceramic flat hair iron.

Step 8. After a period of about 24 hours, the ISR index value of the heat-assisted curl minimized straightened hair was determined as described in Method I. The index value was about 0.878.

Study II (Curl Minimized, Reduction-Oxidation)

The procedure of Study I was repeated, except that in Step 3, no heat was applied to the wrapped tress. The index value determined was about 0.856.

Study III (Comparative)

Step 1. The procedure of Step 2 of Study I was performed to provide reduced hair, except that the viscous, thiol salt-containing, hair-reducing composition was a commercial product of similar strength but containing no PVA (FERM® Conditioning Retexturizing Creme for normal hair, Avlon Industries, Inc., containing about 8.5% ammonium thioglycolate, about 0.4% diammonium dithiodiglycolate, as well as conditioners, emulsifiers, and water, and having a pH in the range of about pH 9.2 to about pH 9.3).

Step 2. The reduced hair was washed with tepid water for about three minutes to remove the hair-reducing composition, washed with shampoo (KERACARE® Hydrating Detangling Shampoo, Avlon Industries, Inc.), and towel blotted.

Step 3. About 0.8 g of conditioner (KERACARE® Leave-In Conditioner, Avlon Industries, Inc.) was applied to the blotted reduced hair from Step 2, and the tress was blow-dried with a hand-held electric hair dryer, set at a high heat setting (about 105° C.) until the hair was substantially dry to the touch.

Step 4. About 0.5 g of a silicone-containing thermal protectant composition (a blend of about one part by weight of KERACARE® Silken Seal (containing silicones) and about one part by weight of FERM® Heat Protection Complex (containing silicone and oils), both available from Avlon Industries, Inc.) was applied to the dry reduced hair, distributed through the hair, and then the tress was pressed by passing the tress three times through with a heated (about 160° C.) flat hair iron.

Step 5. The ironed reduced hair was then neutralized by applying about 2 g of oxidative neutralizer lotion (FERM® Conditioning Neutralizing Lotion, Avlon Industries, Inc., containing sodium bromate and conditioners, and having a pH of about 7) to the hair, distributing the neutralizer through the hair and leaving it in contact with the hair for a period of about 15 minutes.

Step 6. The neutralized tress from Step 5 was thoroughly washed with warm water, and towel blotted.

Step 7. About 0.5 g of conditioner (KERACARE® Leave-In Conditioner, Avlon Industries, Inc.) was applied to the blotted neutralized hair and distributed through the hair.

Step 8. After a period of about 24 hours, the ISR index value was determined. The ISR index value was about 0.823.

The index value data show that the heat-assisted curl minimizing reduction-oxidation process of Study I was the most protective of hair tensile strength, followed by the curl minimizing reduction-oxidation process of Study II, and both were more protective than the comparative reduction-oxidation process of Study III.

Overall, the results show that the curl minimizing agents of this invention can be used to effectively minimize the natural curl in the hair to achieve straightening effects that are comparable to or greater than those obtained with formaldehyde and avoid the negative physiological aspects attendant with the use of formaldehyde. Additionally, the results show that the curl minimizing agents can augment and enhance the permanency of the straightening effect achieved with conventional chemical reduction-oxidation processes.

The foregoing examples are intended to be illustrative and not limiting. Numerous variations and modifications may be effected without departing from the true spirit and scope of the invention.

1. A keratin-fiber protectant, curl minimizing composition comprising an effective keratin-protective amount of a physiologically tolerable curl minimizing agent selected from the group consisting of a polyfunctional aldehyde, an activated olefin-containing compound, and a polycarboxylic acid.

2. The protectant, curl minimizing composition of claim 1 wherein the polyfunctional aldehyde is a dialdehyde selected from the group consisting of glutaraldehyde, and glyoxal.

3. The protectant, curl minimizing composition of claim 1 wherein the activated olefin-containing compound is selected from the group consisting of maleic acid and esters thereof, maleimides and N-substituted derivatives thereof, maleamic acid and N-substituted derivatives thereof, and maleic anhydride

4. The protectant, curl minimizing composition of claim 1 wherein the activated olefin-containing compound comprises at least one poly(C₂-C₄) alkylene glycol ester of maleic acid.

5. The protectant, curl minimizing composition of claim 1 wherein the activated olefin-containing compound comprises poly(oxy-1,2-ethanediyl)-alpha-hydro-omega-hydroxy, (2Z)-2-butenedioate.

6. The protectant, curl minimizing composition of claim 1 wherein the polycarboxylic acid selected from the group consisting of 1,2,3,4-butanetetracarboxylic acid (BTCA), 1,2,3,4-cyclopentanetetracarboxylic (CPTA), citric acid, and polyacrylic acid.

7. The protectant, curl minimizing composition of claim 1 wherein the composition has a pH in the range of about 2 to about 11.

8. The protectant curl minimizing composition of claim 7 wherein the composition includes a Lewis acid catalyst.

9. The protectant curl minimizing composition of claim 1 wherein the composition includes a polyvinyl alcohol.

10. The protectant curl minimizing composition of claim 1 wherein the composition includes a cyclodextrin.

11. A method of minimizing the natural curl in proteinaceous keratin, the method comprising contacting the keratin with a protectant, curl minimizing composition of claim 1 for a time period sufficient to minimize the curl configuration, said contact being optionally heat-assisted.

12. The method of claim 11 further comprising washing the keratin fibers after contact with the protectant curl minimizing composition.

13. (canceled)

14. The method of claim wherein the washed keratin fibers are subsequently contacted with the same or a different one of said curl minimizing composition, said subsequent contact optionally being heat-assisted.

15. (canceled)

16. A method of minimizing the natural curl in proteinaceous keratin, the method comprising the steps of:
(a) contacting the keratin with a keratin-reducing composition for a time period sufficient to produce reduced keratin;
(b) removing the keratin-reducing composition from the reduced keratin by washing with water;
(c) contacting the washed reduced keratin with a protectant, curl minimizing composition of claim 1 for a time period sufficient to minimize the curl configuration, said contact optionally being heat-assisted;
(d) contacting the curl-minimized, reduced keratin with an oxidizing composition for a time period sufficient to oxidize the reduced keratin; and
(e) removing the oxidizing composition from the keratin by washing with water to provide curl-minimized keratin.

17. (canceled)

18. The method of claim 16 wherein after step (e), the curl-minimized keratin is contacted with a subsequent curl minimizing composition that is the same or different than said curl minimizing composition of step (c) and substantially dried, said drying being optionally heat-assisted.

19. The method of claim 18 wherein the subsequent curl-minimizing composition is a thermal protectant composition.

20. (canceled)

21. The method of claim 19 wherein the substantially dry keratin is pressed with a flat iron heated to a temperature of about 160° C.

22. The method of claim 16 performed on human hair keratin.

23. An article of manufacture for use in minimizing the natural curl in human hair comprising a kit containing at least one keratin-fiber protectant, curl minimizing composition of claim 1 in packaged form, and optionally further including at least one of the following:
a preconditioning composition;
a thermal protectant composition, each of the foregoing compositions being in separately packaged form;
instructional indicia for performing a curl minimizing process; and
implements for performing one or more steps in the process.

24. (canceled)

25. The article of manufacture of claim 23 wherein any one of the compositions includes a keratin-protective, curl minimizing agent.

26. The article of manufacture of claim 23 including at least one of the following:
a thiol salt-containing, keratin-reducing composition,
an oxidative composition,
each of the foregoing compositions being in separately packaged form.

27. The method of claim 11 including the step of characterizing the curl minimization by performing Fourier transform analysis of the orientation of the fiber alignment and numerically quantifying the deviation therefrom.
